(19) United States
(12) Patent Application Publication
(10) Pub. No.: US 2022/0088403 A1
(43) Pub. Date: Mar. 24, 2022

(54) Methods And Compositions For Treating Coronavirus

(71) Applicant: NOVOCURE GMBH, Root D4 (CH)

(72) Inventors: Tali Voloshin-Sela, Haifa (IL); Lilach Avigdor, Haifa (IL); Yaara Porat, Haifa (IL); Moshe Giladi, Haifa (IL); Helena Are Yaara, Haifa (IL); Helena Mumblat, Haifa (IL)

(21) Appl. No.: 17/478,174

(22) Filed: Sep. 17, 2021

Related U.S. Application Data
(60) Provisional application No. 63/080,101, filed on Sep. 18, 2020.
(60) Provisional application No. 63/083,456, filed on Sep. 25, 2020.
(60) Provisional application No. 63/126,290, filed on Dec. 16, 2020.

(51) Int. Cl. A61N 1/40; A61N 1/36

(52) U.S. Cl. CPC A61N 1/40; A61N 1/3601

(57) ABSTRACT

Disclosed are methods of inhibiting a coronavirus from infecting or replicating in a cell comprising exposing the cell to an alternating electric field for a period of time, the alternating electric field having a frequency and field strength, wherein the frequency and field strength of the alternating electric field inhibits coronavirus infection or replication. Disclosed are methods of reducing coronavirus copy number per cell comprising exposing the cell to an alternating electric field for a period of time, the alternating electric field having a frequency and field strength, wherein the frequency and field strength of the alternating electric field reduces coronavirus copy number in the cell. Disclosed are methods of treating a subject infected with coronavirus comprising applying an alternating electric field to a target site of the subject for a period of time, the alternating electric field having a frequency and field strength, wherein the target site comprises one or more coronavirus infected cells.

### CROSS-REFERENCE TO RELATED APPLICATIONS

This application claims the benefit of U.S. Provisional Patent Application No. 63/080,101, filed on Sep. 18, 2020, U.S. Provisional Patent Application No. 63/083,456, filed on Sep. 25, 2020, and U.S. Provisional Patent Application No. 63/126,290, filed on Dec. 16, 2020, each of which is incorporated by reference herein in its entirety.

### BACKGROUND

Viruses are small intracellular obligate parasites. Viruses include a nucleic acid that contains the genetic information necessary to program the synthetic machinery of the host cell for viral replication, and, in the simplest viruses, a protective protein coat.

To infect a cell, the virus must attach to the cell surface, penetrate into the cell, and become sufficiently uncoated to make its genome accessible to viral or host machinery for transcription or translation. Viruses' multiplication usually causes cell damage or death. Productive infection results in the formation of progeny viruses.

Coronaviruses are a group of RNA viruses that cause diseases in mammals and birds. In humans and birds, they cause respiratory tract infections that can range from mild to lethal. Mild illnesses in humans include some cases of the common cold (which is also caused by other viruses, predominantly rhinoviruses), while more lethal varieties can cause SARS, MERS, and COVID-19. In cows and pigs they cause diarrhea, while in mice they cause hepatitis and encephalomyelitis.

Coronaviruses are members of the subfamily Orthocoronavirinae, in the family Coronaviridae, order Nidovirales, and realm Riboviria. They are enveloped viruses with a positive-sense single-stranded RNA genome and a nucleocapsid of helical symmetry. The genome size of coronaviruses ranges from approximately 26 to 32 kilobases, one of the largest among RNA viruses. They have characteristic club-shaped spikes that project from their surface, which in electron micrographs create an image reminiscent of the solar corona, from which their name derives.

Over the past two decades, emerging pathogenic coronaviruses capable of causing life-threatening disease in humans and animals have been identified, namely severe acute respiratory syndrome coronavirus (SARS-CoV) and Middle Eastern respiratory syndrome coronavirus (MERS-CoV). In December 2019, the Wuhan Municipal Health Committee (Wuhan, China) identified an outbreak of viral pneumonia cases of unknown cause. Coronavirus RNA was identified in some of these patients. This novel coronavirus has been named SARS-CoV-2, and the disease caused by this virus has been named COVID-19. Currently there are approximately 50 million confirmed cases of COVID-19 and over 1.2 million deaths globally.

Individuals of all ages are at risk for infection and severe disease. However, the probability of serious COVID-19 disease is higher in people aged >60 years, those living in a nursing home or long-term care facility, and those with chronic medical conditions. The spectrum of illness can range from asymptomatic infection to severe pneumonia with acute respiratory distress syndrome (ARDS) and death. Although COVID-19 patients can present with many different symptoms the main symptoms are fever, cough or shortness of breath. The abnormalities seen in chest X-rays vary, but bilateral multi-focal opacities are the most common. The abnormalities seen in computed tomography (CT) of the chest also vary, but the most common are bilateral peripheral ground-glass opacities, with areas of consolidation developing later in the clinical course. In the early phase of the disease and in an asymptomatic presentation the imaging of both X-ray and CT can be normal. Virologic testing (i.e., using a molecular diagnostic or antigen test to detect SARS-CoV-2) is recommended by the NIH for diagnosing SARS-CoV-2 in patients with suspected COVID-19 symptoms.

COVID-19 patients can be grouped into the following groups by illness severity-asymptomatic or presymptomatic, mild, moderate, severe and critical illness, where patients with severe illness are individuals who have respiratory frequency >30 breaths per minute, SpO2<94% on room air at sea level, ratio of arterial partial pressure of oxygen to fraction of inspired oxygen (PaO2/FiO2)<300 mmHg, or lung infiltrates >50%. The management of a COVID-19 patient with severe illness includes pulmonary imagining and ECG, if indicated. Laboratory evaluation includes a complete blood count (CBC) with differential and a metabolic profile, including liver and renal function tests. Measurements of inflammatory markers such as C-reactive protein (CRP), D-dimer, and ferritin, while not part of standard care, may have prognostic value.

Although it has been almost a year since the first case of COVID-19 pneumonia, current treatment options are limited and involve the treatment of symptoms, supportive care, isolation, and experimental measures. Therefore, there is an urgent unmet need to develop new therapies for the treatment of COVID-19.

### BRIEF SUMMARY

It has previously been shown that when cells are exposed to an alternating electric field (AEF) in specific frequency ranges while the cell is undergoing mitosis, the AEF can disrupt the mitosis process and cause apoptosis. This phenomenon has been successfully used to treat tumors (e.g. glioblastoma, mesothelioma, etc.) as described in U.S. Pat. Nos. 7,016,725 and 7,565,205, each of which is incorporated herein by reference in its entirety. And in the context of treating tumors, these alternating electric fields are referred to as “TTFields” (or “Tumor Treating Fields”). One of the reasons why TTFields therapy is well-suited for treating tumors is that TTFields selectively disrupt dividing cells during mitosis, and apparently have no effect on cells that are not dividing. And because tumor cells divide much more often than other cells in a person's body, applying TTFields to a subject will selectively attack the tumor cells while leaving the other cells unharmed. The same phenomenon has also been successfully shown to be useful for destroying bacteria, as described in U.S. Pat. No. 9,750,934, which is incorporated herein by reference in its entirety. And here again, one of the reasons why this approach is well-suited for destroying bacteria is that bacteria cells divide much more rapidly than other cells in a person's body.

Disclosed herein are methods of inhibiting a coronavirus from infecting or replicating in a cell comprising: exposing the cell to an alternating electric field for a period of time, the alternating electric field having a frequency and field strength, wherein the frequency and field strength of the alternating electric field inhibits coronavirus infection or replication.

Disclosed herein are methods of reducing coronavirus copy number per cell comprising: exposing the cell to an alternating electric field for a period of time, the alternating electric field having a frequency and field strength, wherein the frequency and field strength of the alternating electric field reduces virus copy number in the cell.

Disclosed herein are methods of treating a subject infected with coronavirus comprising: applying an alternating electric field to a target site of the subject for a period of time, the alternating electric field having a frequency and field strength, wherein the target site comprises one or more coronavirus infected cells.

Disclosed are methods of treating a subject at risk for infection with coronavirus comprising applying an alternating electric field to a target site of the subject for a period of time, the alternating electric field having a frequency and field strength.

Disclosed are methods of preventing the spread of coronavirus from a subject infected with a coronavirus to a non-infected subject comprising: applying an alternating electric field to a target site of the subject infected with a coronavirus for a period of time, the alternating electric field having a frequency and field strength, wherein the target site comprises one or more coronavirus infected cells

Disclosed are methods of preventing coronavirus infection in a subject comprising: applying an alternating electric field to a target site of the subject for a period of time, the alternating electric field having a frequency and field strength.

Disclosed are methods of reducing replication of a coronavirus in a cell comprising: exposing the cell to an alternating electric field for a period of time, the alternating electric field having a frequency and field strength, wherein the frequency and field strength of the alternating electric field reduces virus copy number in the cell.

Additional advantages of the disclosed method and compositions will be set forth in part in the description which follows, and in part will be understood from the description, drawings, and/or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows an example of an effect of TTFields application for 24 hours during the infection and replication phases on cell proliferation and virus replication.

FIG. 2 shows an example of an effect of TTFields application for 48 hours during the infection and replication phases on cell proliferation and virus replication.

FIG. 3 shows an example of cell growth with and without TTFields in the absence of virus.

FIG. 4 shows an example of an effect of TTFields application on cell count and on virus copies during infection only (TTFields+Infection) or during infection and replication phases. (TTFields+Infection+Proliferation).

FIG. 5 shows an example of 229E copy number/cell and PR8 copy number/cell.

FIG. 6 shows an example of an effect of TTFields on 229E depending on viral concentration and time. In all experiments with virus, the control cells are infected cells that do not receive TTFields.

FIG. 7 shows an example of an effect of 48 hr TTFields treatment on PR8 virus in A549 cells.

FIG. 8 shows an effect of 48 hours TTFields application to MRC5 cells infected with the 229E virus during the viral infection and proliferation on concentrations of replication competent lytic virions (PFU).

FIG. 9 is an example experimental design for treating cells infected with virus with TTFields.

FIGS. 10A-10F show an exemplary effect of TTFields on viral entry. This was done as a frequency scan to evaluate optimal frequency. (FIG. 10A) MRC-5 cells were infected with 1% HCoV-229E virus while being exposed to TTFields at 100, 150 or 400 kHz, and cellular viral load measured by RT-qPCR at 2 hpi (hours post infection). (FIG. 10B) MRC5 cells were infected with 1% HCoV-229E virus while being exposed to 150 kHz TTFields, followed by cellular viral load measured by RT-qPCR at 0.5 hpi. (FIG. 10C and FIG. 10D) MRC-5 cells were infected with HCoV-229E virus at a multiplicity of infection (MOI) of 20 while being exposed to 150 kHz TTFields, followed by SEM examination at 0.5 hpi to determine the number of viruses (designated with yellow arrows) attached to the cells. (e and f) MRC-5 cells were infected with 3% HCoV-229E virus for 3 hr, the cells were washed and only then TTFields was applied. At 48 hpi, TEM examination were performed to determine the distance of the viruses (designated with white arrows, N=150 per group) from the cells. RQ, relative quantification; ER=endoplasmic reticulum; DMSs=double-membrane spherules; black asterisks=double-membrane vesicles (DMVs). Values are mean±SD. *p<0.05, ***p<0.001, and ****p<0.0001 relative to control; One-way ANOVA for a; Student's T-test for c; Mann-Whitney test for e.

FIGS. 11A-11B and 11C show an exemplary effect of TTFields on long-term viral exposure. MRC-5 cells were infected with 0.01% HCoV-229E virus for 3 hr while being exposed to TTFields for 24, 48 or 72 hr, and the intracellular (FIG. 11A) and extracellular (FIG. 11B) viral amount was examined by RT-qPCR. Cell count was also measured (FIG. 11C). RQ=relative quantification, ns=non-significant. Values are mean±SD. *p<0.05, **p<0.01, and ***p<0.001 relative to control; Sidak's multiple comparison.

FIGS. 12A-12K show an exemplary effect of TTFields on viral replication. (FIGS. 12A-12D) MRC-5 cells were infected with 1% HCoV-229E virus for 3 hr, the cells were washed and only then TTFields were applied. At 24 hpi, dsRNA was detected by fluorescent microscopy with green staining, and cellular nuclei imaged with blue DAPI (×20 magnification). The number of foci per infected cell, foci size, and foci area per infected cell were quantified. (FIGS. 12E-121) MRC-5 cells were infected with 3% HCoV-229E virus for 3 hr, the cells were washed and only then TTFields were applied. At 48 hpi, TEM examination were performed to measure invaginations (designated with black arrows) and fusion (designated with yellow arrows) of double-membrane vesicles (DMVs), and to quantify autophagolysosomes (designated with white arrows). Black asterisks=DMVs; White asterisks=intracellular viral particles within a membrane-bound vacuole; AL=autophagolysosome; M=mitochondria; Lys=lysosome. (FIG. 12J and FIG. 12K) Supernatants from 48 hr long-term exposure experiments were added to MRC-5 cells (that were not exposed to TTFields at any stage), and plaque formation was determined for equal virus numbers, or calculated for equal supernatant volume. PFU=plaque forming units; SN=supernatant. Values are mean±SD. *p<0.05, **p<0.01, ***p<0.001, and ****p<0.0001 relative to control; Student's T-test.

FIGS. 13A-13C show an example of the combined effect of TTFields with remdesivir. MRC-5 cells were infected with 1% HCoV-229E virus for 3 hr while being exposed for 48 hr to TTFields, alone or concomitant 0.011 or 0.023 μM Remdesivir, and the intracellular (FIG. 13A) and extracellular (FIG. 13B) viral amount was examined by RT-qPCR. dsRNA was detected by fluorescent microscopy with green staining, and cellular nuclei imaged with blue DAPI (×20 magnification) (FIG. 13C). RQ=relative quantification. Values are mean±SD. **p<0.01, ***p<0.001, and ****p<0.0001 relative to control; Sidak's multiple comparison.

FIG. 14 shows an example of a clinical study design.

FIG. 15 shows an example of virus excretion levels over time. MRC-5 cells were infected with 0.01% HCoV-229E virus for 3 hr, and the extracellular viral amount was examined by RT-qPCR at 24 and 48 hpi. SN=supernatant.

FIG. 16 shows an exemplary effect of TTFields on MRC-5 cells with no virus. MRC-S cells were exposed to TTFields for 24 or 48 hr, and cell count was measured. Values are mean±SD. Student's T-test, relative to control; ns=non-significant. Sidak's multiple comparison.

FIG. 17A-17C shows exemplary effects of TTFields on A549 viral infection. A549 cells were infected with 0.01% HCoV-229E virus for 3 hr while being exposed to TTFields for 48 hr, and the intracellular (FIG. 17A) and extracellular (FIG. 17B) viral amount was examined by RT-qPCR. Cell count was also measured with or without viral infection (FIG. 17C). RQ=relative quantification, ns=non-significant, w/o=without. Values are mean±SD. ***p<0.001, and ****p<0.0001 relative to control; Student's T-test for FIG. 17A and FIG. 17B; Sidak's multiple comparison for FIG. 17C.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a cell” includes a plurality of such cells, reference to “the cell” is a reference to one or more cells and equivalents thereof known to those skilled in the art, and so forth.

As used herein, “coronavirus” refers to a group of RNA viruses of the subfamily Orthocoronavirinae, in the family Coronaviridae, order Nidovirales, and realm Riboviria. They are enveloped viruses with a positive-sense single-stranded RNA genome and a nucleocapsid of helical symmetry. The genome size of coronaviruses ranges from approximately 26 to 32 kilobases, one of the largest among RNA viruses. They have characteristic club-shaped spikes that project from their surface, which in electron micrographs create an image reminiscent of the solar corona, from which their name derives. In some aspects, the coronavirus is Middle East respiratory syndrome coronavirus (MERS-CoV), Human Coronavirus—Erasmus Medical Centre (HCoV-EMC), SARS-CoV, or SARS-CoV-2.

As used herein, a “target site” is a specific site or location within or present on a subject or patient. For example, a “target site” can refer to, but is not limited to a cell, population of cells, an organ, or tissue. In some aspects, the target site can be a cell infected with or comprising a coronavirus. In some aspects, the organ can be the lungs. In some aspects, a cell or population of cells can be one or more lung cells. In some aspects, a “target site” can be a lung cell target site. In some aspects, the target site can be the nasal cavity or nasopharynx. In some aspects, a “target site” can be a specific site or location where coronavirus receptors are present. In some aspects, a “target site” can be a specific site or location where SARS-CoV-2 receptors, particularly the ACE2 receptor, are present. In some aspects, a “target site” can be a specific site or location where dipeptidyl peptidase-4 (DPP-4) or aminopeptidase N (APN) are present.

As used herein, an “alternating electric field” or “alternating electric fields” refers to a very-low-intensity, directional, intermediate-frequency alternating electrical fields delivered to a subject, a sample obtained from a subject or to a specific location within a subject or patient (e.g. a target site). In some aspects, the alternating electrical field can be in a single direction or multiple directional. In some aspects, alternating electric fields can be delivered through two pairs of transducer arrays that generate perpendicular fields within the treated lung. For example, for the Optune™ system (an alternating electric fields delivery system) one pair of electrodes is located to the left and right (LR) of the lung, and the other pair of electrodes is located anterior and posterior (AP) to the lung. Cycling the field between these two directions (i.e., LR and AP) ensures that a maximal range of cell orientations is targeted.

In-vivo and in-vitro studies show that the efficacy of alternating electric fields therapy increases as the intensity of the electrical field increases. Therefore, optimizing array placement on the patient to increase the intensity in the desired region can be performed with the Optune system. Array placement optimization may be performed by “rule of thumb” (e.g., placing the arrays on the chest as close to the desired region of the target site (e.g. lungs) as possible), measurements describing the geometry of the patient's body, body dimensions. Measurements used as input may be derived from imaging data. Imaging data is intended to include any type of visual data. In certain implementations, image data may include 3D data obtained from or generated by a 3D scanner (e.g., point cloud data). Optimization can rely on an understanding of how the electrical field distributes within the lung as a function of the positions of the array and, in some aspects, take account for variations in the electrical property distributions within the target site (e.g. infected cell) of different patients.

The term “subject” refers to the target of administration, e.g. an animal. Thus, the subject of the disclosed methods can be a vertebrate, such as a mammal. For example, the subject can be a human. The term does not denote a particular age or sex. Subject can be used interchangeably with “individual” or “patient.” For example, the subject of administration can mean the recipient of the alternating electrical field.

By “treat” is meant to administer or apply a therapeutic, such as alternating electric fields, to a subject, such as a human or other mammal (for example, an animal model), that has a coronavirus infection, is at risk of being infected with a coronavirus, or has an increased susceptibility for developing a coronavirus infection, in order to prevent or delay a worsening of the effects of the coronavirus infection, or to partially or fully reverse the effects of the coronavirus infection. For example, treating a subject infected with coronavirus can comprise inhibiting a coronavirus from infecting or replicating in a cell in the subject or reducing coronavirus copy number per cell in a subject.

By “prevent” is meant to minimize or decrease the chance that a subject will develop a coronavirus infection.

As used herein, the terms “administering” and “administration” refer to any method of providing a therapeutic, such as an antiviral agent or coronavirus therapeutic (e.g., remdesivir or plasma therapy), to a subject. Such methods are well known to those skilled in the art and include, but are not limited to: oral administration, transdermal administration, administration by inhalation, nasal administration, topical administration, intravaginal administration, ophthalmic administration, intramural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration so as to treat a subject. In some aspects, administering comprises exposing. Thus, in some aspects, exposing a subject to alternating electrical fields means administering alternating electrical fields to the subject.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods of Inhibiting a Coronavirus Infection or Replication

In some aspects, the disclosed methods of inhibiting infection or inhibiting replication of a virus in a cell can be used for any virus that relies on an electrostatic interaction with a receptor. For example, the virus can be a coronavirus or lentivirus. Described herein the coronavirus is used as an example of a virus that can be inhibited from infecting or replicating in a cell. In some aspects, the disclosed methods of inhibiting infection or inhibiting replication of a virus in a cell can be used for any virus that relies on an electrostatic interaction with a receptor, wherein the virus is not influenza.

Disclosed are methods of inhibiting a coronavirus from infecting or replicating in a cell comprising exposing the cell to an alternating electric field for a period of time, the alternating electric field having a frequency and field strength, wherein the frequency and field strength of the alternating electric field inhibits coronavirus infection or replication. In some aspects, the coronavirus is Middle East respiratory syndrome coronavirus (MERS-CoV), Human Coronavirus-Erasmus Medical Centre (HCoV-EMC), SARS-CoV, or SARS-CoV-2. In some aspects, a coronavirus can be an alphacoronavirus, betacoronavirus, gammacoronavirus, or deltacoronavirus. Examples of alphacoronaviruses can include, but are not limited to, Alphacoronavirus 1, Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512. Examples of betacoronaviruses can include, but are not limited to, Betacoronavirus 1 (Bovine Coronavirus, Human coronavirus OC43), Hedgehog coronavirus 1, Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus (SARS-CoV, SARS-CoV-2), Tylonycteris bat coronavirus HKU4. Examples of gammacoronaviruses can include, but are not limited to, Avian coronavirus, Beluga whale coronavirus SW1. Examples of deltacoronaviruses can include, but are not limited to, Bulbul coronavirus HKU11, Porcine coronavirus HKU15. In some aspects, the coronavirus can be a variant of one of more coronaviruses. For example a variant can be a variant of SARS-CoV-2, such as the Alpha (B.1.1.7), Beta (B.1.351, B.1.351.2, B.1.351.3), Delta (B.1.617.2, AY.1, AY.2, AY.3), and Gamma (P.1, P.1.1, P.1.2) variants.

In some aspects, the frequency of the alternating electric fields is 150 kHz.

In some aspects, the parameters of the alternating electric fields is 150 kHz and 1.7 V/cm.

In some aspects, the alternating electric field is applied at a frequency of between 250 kHz and 350 kHz. In some aspects, the alternating electric field is applied at a frequency between 50 and 190 kHz. In some aspects, the alternating electric field is applied at a frequency between 210 and 400 kHz. In some aspects, the alternating electric field is applied at a frequency between 50 kHZ and 1 MHz. In some aspects, the alternating electric field has a field strength of at least 1 V/cm RMS. In some aspects, the alternating electric field has a frequency between 50 and 190 kHz. In some aspects, the alternating electric field has a frequency between 210 and 400 kHz. In some aspects, the alternating electric field has a field strength of at least 1 V/cm RMS. In some aspects, the alternating electric field has a field strength between 1 and 4 V/cm RMS.

In some aspects, the period of time can be hours, days, or weeks. In some aspects, cells can be exposed to an alternating electric field for 24 or 48 hours.

In some aspects, the alternating electric field inhibits both coronavirus infection and replication. In some aspects, the alternating electric field inhibits coronavirus infection. In some aspects, the alternating electric field inhibits coronavirus replication.

In some aspects, the alternating electric field promotes fusion of an authophagosome with a lysosome resulting in the lysis of the virus.

In some aspects, the alternating electric field can prevent or reduce the amount of virus being shed by a cell. Thus, in some aspects, the alternating electric field can result in infected cells producing less virus which then results in less re-infection.

In some aspects, the cells not infected with a coronavirus are not damaged.

In some aspects, the cell viability is maintained.

In some aspects, the viral load in the subject is decreased and cell proliferation is unaffected.

In some aspects, the viability of the cells at the target site is maintained and viral replication or viral infection is decreased.

Alternating electric fields are known to induce an anti-mitotic effect by exerting bi-directional forces on highly polar intracellular elements, such as tubulin. Thus, in some aspects, alternating electric fields could have an effect on other highly polar elements, such as viral proteins. For example, the spike proteins of a coronavirus are highly polar and electrostatic allowing for binding to its receptor (e.g. ACE2). In some aspects, the alternating electric fields can interfere with the ability of the coronavirus to interact with its receptor. Interfering with the ability of the coronavirus to interact with its receptor can disrupt or inhibit the ability of the coronavirus to infect a cell.

In some aspects, alternating electric fields can prevent a virus from infecting a cell. For example, in some aspects, alternating electric fields can prevent or decrease a virus from getting close enough to a cell membrane to infect the cell.

In some aspects, alternating electric fields can increase the fusion of a virus with a lysosome. The fusion of a virus with a lysosome can result in killing of the virus. In some aspects, the virus is a coronavirus and alternating electric fields can increase the fusion of a coronavirus with a lysosome.

### C. Methods of Reducing Virus Copy Number

In some aspects, the disclosed methods of reducing copy number of a virus in a cell can be used for any virus that relies on electrostatic interaction with a receptor. For example, the virus can be a coronavirus or lentivirus. As described herein, a coronavirus is used as an example of a virus in which copy number can be reduced by application of an alternating electrical field.

Disclosed are methods of reducing coronavirus copy number per cell comprising exposing the cell to an alternating electric field for a period of time, the alternating electric field having a frequency and field strength, wherein the frequency and field strength of the alternating electric field reduces virus copy number in the cell.

Also disclosed are methods of reducing replication of coronavirus in a cell comprising exposing the cell to an alternating electric field for a period of time, the alternating electric field having a frequency and field strength, wherein the frequency and field strength of the alternating electric field reduces virus copy number in the cell. In some aspects, the coronavirus is Middle East respiratory syndrome coronavirus (MERS-CoV), Human Coronavirus-Erasmus Medical Centre (HCoV-EMC), SARS-CoV, or SARS-CoV-2. In some aspects, a coronavirus can be an alphacoronavirus, betacoronavirus, gammacoronavirus, or deltacoronaviruses. Examples of alphacoronaviruses can include, but are not limited to, Alphacoronavirus 1, Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512. Examples of betacoronavirus can include, but are not limited to, Betacoronavirus 1 (Bovine Coronavirus, Human coronavirus OC43), Hedgehog coronavirus 1, Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus (SARS-CoV, SARS-CoV-2), Tylonycteris bat coronavirus HKU4. Examples of gammacoronaviruses can include, but are not limited to, Avian coronavirus, Beluga whale coronavirus SW1. Examples of deltacoronaviruses can include, but are not limited to, Bulbul coronavirus HKU11, Porcine coronavirus HKU15. In some aspects, the coronavirus can be a variant of one of more coronaviruses. For example a variant can be a variant of SARS-CoV-2, such as the Alpha (B.1.1.7), Beta (B.1.351, B.1.351.2, B.1.351.3), Delta (B.1.617.2, AY.1, AY.2, AY.3), and Gamma (P.1, P.1.1, P.1.2) variants.

In some aspects, the frequency of the alternating electric fields is 150 kHz.

In some aspects, the parameters of the alternating electric fields is 150 kHz and 1.7 V/cm.

In some aspects, the alternating electric field is applied at a frequency of between 250 kHz and 350 kHz. In some aspects, the alternating electric field is applied at a frequency between 50 and 190 kHz. In some aspects, the alternating electric field is applied at a frequency between 210 and 400 kHz. In some aspects, the alternating electric field is applied at a frequency between 50 kHz and 1 MHz. In some aspects, the alternating electric field has a field strength of at least 1 V/cm RMS. In some aspects, the alternating electric field has a frequency between 50 and 190 kHz. In some aspects, the alternating electric field has a frequency between 210 and 400 kHz. In some aspects, the alternating electric field has a field strength of at least 1 V/cm RMS. In some aspects, the alternating electric field has a field strength between 1 and 4 V/cm RMS.

In some aspects, the period of time can be hours, days, or weeks. In some aspects, cells can be exposed to an alternating electric field for 24 or 48 hours.

In some aspects, a reduction in coronavirus copy number per cell is determined based on a comparison to coronavirus copy number per cell in cells not treated with an alternating electric field.

In some aspects, a reduction in coronavirus copy number per cell is achieved while simultaneously maintaining cell viability.

In some aspects, the cells not infected with a coronavirus are not damaged.

In some aspects, the cell viability is maintained.

In some aspects, the viral load in the subject is decreased and cell proliferation is unaffected.

In some aspects, the viability of the cells at the target site is maintained and viral replication or viral infection is decreased.

### D. Methods of Treating

In some aspects, the disclosed methods of treating a subject infected with a virus can be used for any virus that relies on electrostatic interaction with a receptor. For example, the virus can be a coronavirus or lentivirus. As described herein, a coronavirus is used as an example of a virus in which alternating electric fields can be used to treat a subject infected with coronavirus.

Disclosed are methods of treating a subject infected with coronavirus comprising applying an alternating electric field to a target site of the subject for a period of time, the alternating electric field having a frequency and field strength, wherein the target site comprises one or more coronavirus infected cells.

Disclosed are methods of treating a subject at risk for infection with coronavirus comprising applying an alternating electric field to a target site of the subject for a period of time, the alternating electric field having a frequency and field strength. In some aspects, a subject at risk for infection with coronavirus can be a first responder (e.g. healthcare worker) or a subject in close contact with subjects known to have coronavirus or to have been exposed to a coronavirus.

Disclosed are methods of preventing coronavirus infection in a subject comprising: applying an alternating electric field to a target site of the subject for a period of time, the alternating electric field having a frequency and field strength. In some aspects, the subject is not previously infected with coronavirus. In some aspects, the subject is at risk of being infected with a coronavirus. In some aspects, the coronavirus is Middle East respiratory syndrome coronavirus (MERS-CoV), Human Coronavirus-Erasmus Medical Centre (HCoV-EMC), SARS-CoV, or SARS-CoV-2. In some aspects, a coronavirus can be an alphacoronavirus, betacoronavirus, gammacoronavirus, or deltacoronavirus. Examples of alphacoronaviruses can include, but are not limited to, Alphacoronavirus 1, Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512. Examples of betacoronaviruses can include, but are not limited to, Betacoronavirus 1 (Bovine Coronavirus, Human coronavirus OC43), Hedgehog coronavirus 1, Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus (SARS-CoV, SARS-CoV-2), Tylonycteris bat coronavirus HKU4. Examples of gammacoronaviruses can include, but are not limited to, Avian coronavirus, Beluga whale coronavirus SW1. Examples of deltacoronaviruses can include, but are not limited to, Bulbul coronavirus HKU11, Porcine coronavirus HKU15. In some aspects, the coronavirus can be a variant of one of more coronaviruses. For example a variant can be a variant of SARS-CoV-2, such as the Alpha (B.1.1.7), Beta (B.1.351, B.1.351.2, B.1.351.3), Delta (B.1.617.2, AY.1, AY.2, AY.3), and Gamma (P.1, P.1.1, P.1.2) variants.

In some aspects, the frequency of the alternating electric fields is 150 kHz. In some aspects, the alternating electric fields is about 150 kHz-300 kHz.

In some aspects, the parameters of the alternating electric fields are 150 kHz and 1.7 V/cm.

In some aspects, the alternating electric field is applied at a frequency of between 250 kHz and 350 kHz. In some aspects, the alternating electric field is applied at a frequency of between 250 kHz and 350 kHz. In some aspects, the alternating electric field is applied at a frequency between 50 and 190 kHz. In some aspects, the alternating electric field is applied at a frequency between 210 and 400 kHz. In some aspects, the alternating electric field is applied at a frequency between 50 kHz and 1 MHz. In some aspects, the alternating electric field has a field strength of at least 1 V/cm RMS. In some aspects, the alternating electric field has a frequency between 50 and 190 kHz. In some aspects, the alternating electric field has a frequency between 210 and 400 kHz. In some aspects, the alternating electric field has a field strength of at least 1 V/cm RMS. In some aspects, the alternating electric field has a field strength between 1 and 4 V/cm RMS.

Disclosed are methods of treating COVID-19 in a subject comprising applying an alternating electric field to a target site of the subject for a period of time, the alternating electric field having a frequency and field strength, wherein the target site comprises one or more SARS-CoV-2 infected cells.

In some aspects, the alternating electric field reduces viral copy number in the one or more coronavirus infected cells. In some aspects, the frequency of the alternating electric fields is 150 kHz. In some aspects, the parameters of the alternating electric fields is 150 kHz and 1.7 V/cm. In some aspects, the alternating electric field is applied at a frequency of between 250 kHz and 350 kHz. In some aspects, the alternating electric field is applied at a frequency between 50 and 190 kHz. In some aspects, the alternating electric field is applied at a frequency between 210 and 400 kHz. In some aspects, the alternating electric field has a field strength of at least 1 V/cm RMS. In some aspects, the alternating electric field has a frequency between 50 and 190 kHz. In some aspects, the alternating electric field has a frequency between 210 and 400 kHz. In some aspects, the alternating electric field has a field strength of at least 1 V/cm RMS. In some aspects, the alternating electric field has a field strength between 1 and 4 V/cm RMS.

In some aspects, the disclosed methods further comprise administering a second therapeutic to the subject. In some aspects, a second therapeutic can be, but is not limited to, an antiviral therapeutic. In some aspects, the second therapeutic is administered prior to the alternating electrical field. In some aspects, the second therapeutic is administered simultaneously to the alternating electrical field. In some aspects, the second therapeutic is administered after the alternating electrical field. In some aspects an antiviral agent is delivered to the subject or target region so that the antiviral agent is present in the target region while the alternating electrical field is administered. In some aspects, the antiviral therapeutic is a cellular or gene therapy therapeutic, an immunomodulatory, an antibody or mixture of antibodies or an antiviral. In some aspects, the antiviral therapeutic is remdesivir (Veklury), Avigan (favilavir), bamlanivimab, Olumiant and Baricinix (baricitinib), hydroxychloroquine/chloroquine, Casirivimab and imdevimab (formerly REGN-COV2), PTC299, Leronlimab (PRO 140), Bamlanivimab (LY-CoV555), Lenzilumab, Ivermectin, RLF-100 (aviptadil), Metformin (Glucophage, Glumetza, Riomet), AT-527, Actemra (tocilizumab), Niclocide (niclosamide), Convalescent plasma, Pepcid (famotidine), Kaletra (lopinavir-ritonavir), Remicade (infliximab), AZD7442, AZD7442, CT-P59, Heparin (UF and LMW), VIR-7831 (GSK4182136), JS016, Kevzara (sarilumab), SACCOVID (CD24Fc), Humira (adalimumab), COVI-GUARD (STI-1499), Dexamethasone (Dextenza, Ozurdex, others), PB1046, Galidesivir, Bucillamine, PF-00835321 (PF-07304814), Eliquis (Apixaban), Takhzyro (lanadelumab), Hydrocortisone, Ilaris (canakinumab), Colchicine (Mitigare, Colcrys), BLD-2660, Avigan (favilavir/avifavir), Rhu-pGSN (gelsolin), MK-4482, TXA127, LAM-002A (apilimod dimesylate), DNL758 (SAR443122), INOpulse, ABX464, AdMSCs, Losmapimod, Mavrilimumab, or Calquence (acalabrutinib). In some aspects, the coronavirus is Middle East respiratory syndrome coronavirus (MERS-CoV), Human Coronavirus-Erasmus Medical Centre (HCoV-EMC), SARS-CoV, or SARS-CoV-2. In some aspects, a coronavirus can be an alphacoronavirus, betacoronavirus, gammacoronavirus, or deltacoronavirus. Examples of alphacoronaviruses can include, but are not limited to, Alphacoronavirus 1, Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512. Examples of betacoronaviruses can include, but are not limited to, Betacoronavirus 1 (Bovine Coronavirus, Human coronavirus OC43), Hedgehog coronavirus 1, Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus (SARS-CoV, SARS-CoV-2), Tylonycteris bat coronavirus HKU4. Examples of gammacoronaviruses can include, but are not limited to, Avian coronavirus, Beluga whale coronavirus SW1. Examples of deltacoronaviruses can include, but are not limited to, Bulbul coronavirus HKU11, Porcine coronavirus HKU15. In some aspects, the coronavirus can be a variant of one of more coronaviruses. For example a variant can be a variant of SARS-CoV-2, such as the Alpha (B.1.1.7), Beta (B.1.351, B.1.351.2, B.1.351.3), Delta (B.1.617.2, AY.1, AY.2, AY.3), and Gamma (P.1, P.1.1, P.1.2) variants.

In some aspects, the target site can be the lungs. In some aspects, the target site is tumor-free.

Also disclosed are methods of preventing the spread of a coronavirus. In some aspects, a subject infected with coronavirus can be treated by applying an alternating electric field to a target site of the subject for a period of time, the alternating electric field having a frequency and field strength, wherein the target site comprises one or more coronavirus infected cells. The alternating electric field can result in production of a defective virus, wherein the defective virus is less infectious than the wild type coronavirus, so that the subject is less infectious to other subjects. In some aspects, the alternating electric field can alter the virus being produced by the infected cells in the subject thereby resulting in a defective virus, or the alternating electric field can alter the cells producing the virus thereby resulting in production of a defective virus. In some aspects, the alternating electric field can render the replication machinery required for coronavirus replication.

In some aspects, cells within the subject that are not infected with a coronavirus are not damaged.

In some aspects, the cell viability in the target site is maintained.

In some aspects, the viral load in the subject is decreased and cell proliferation is unaffected.

In some aspects, the viability of the cells at the target site is maintained and viral replication or viral infection is decreased.

### E. Combination Therapy

Any of the disclosed methods of using TTFields can be performed in combination with one or more of the known standards of care for coronavirus infection. Thus, in some aspects, TTFields can be combined with an antibody, or antibody cocktail, nanobody, antiviral small molecules, macromolecules of sulfated polysaccharides, and polypeptides. Frequent targets are the viral spike protein, the host angiotensin converting enzyme 2, the host transmembrane protease serine 2, and clathrin-mediated endocytosis. For example, disclosed methods of using TTFields can be performed in combination with one or more of remdesivir (Veklury), Nafamostat, Avigan (favilavir), bamlanivimab, Olumiant and Baricinix (baricitinib), hydroxychloroquine/chloroquine, Casirivimab and imdevimab (formerly REGN-COV2), PTC299, Leronlimab (PRO 140), Bamlanivimab (LY-CoV555), Lenzilumab, Ivermectin, RLF-100 (aviptadil), Metformin (Glucophage, Glumetza, Riomet), AT-527, Actemra (tocilizumab), Niclocide (niclosamide), Convalescent plasma, Pepcid (famotidine), Kaletra (lopinavir-ritonavir), Remicade (infliximab), AZD7442, AZD7442, CT-P59, Heparin (UF and LMW), VIR-7831 (GSK4182136), JS016, Kevzara (sarilumab), SACCOVID (CD24Fc), Humira (adalimumab), COVI-GUARD (STI-1499), Dexamethasone (Dextenza, Ozurdex, others), PB1046, Galidesivir, Bucillamine, PF-00835321 (PF-07304814), Eliquis (Apixaban), Takhzyro (lanadelumab), Hydrocortisone, Ilaris (canakinumab), Colchicine (Mitigare, Colcrys), BLD-2660, Avigan (favilavir/avifavir), Rhu-pGSN (gelsolin), MK-4482, TXA127, LAM-002A (apilimod dimesylate), DNL758 (SAR443122), INOpulse, ABX464, AdMSCs, Losmapimod, Mavrilimumab, or Calquence (acalabrutinib), quinoline-based antimalarials ((hydroxy)-chloroquine and others), RAAS modifiers (captopril, losartan, and others), statins (atorvastatin and simvastatin), guanidino-based serine protease inhibitors (camostat and nafamostat), antibacterials (macrolides, clindamycin, and doxycycline), antiparasitics (ivermectin and niclosamide), cardiovascular drugs (amiodarone, verapamil, and tranexamic acid), antipsychotics (chlorpromazine), antivirals (umifenovir and oseltamivir), DPP-4 inhibitors (linagliptin), JAK inhibitors (baricitinib and others), sulfated glycosaminoglycans (UFH and LMWHs) and polypeptides such as the enzymes DAS181 and rhACE2. They also include the viral spike protein-targeting monoclonal antibodies REGN10933 and REGN10987.

### F. Alternating Electric Fields

The methods disclosed herein comprise alternating electric fields. In some aspects, the alternating electric field used in the methods disclosed herein is a tumor-treating field. In some aspects, the alternating electric field can vary dependent on the type of cell or condition to which the alternating electric field is applied. In some aspects, the alternating electric field can be applied through one or more electrodes placed on the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the frequency of the alternating electric fields can be 150 kHz. The frequency of the alternating electric fields can also be, but is not limited to, about 150 kHz, about 200 kHz, between 50 and 500 kHz, between 100 and 500 kHz, between 25 kHz and 1 MHz, between 50 kHz and 1 MHz, between 50 and 190 kHz, between 25 and 190 kHz, or between 210 and 400 kHz. In some aspects, the frequency of the alternating electric fields can be electric fields at 50 kHz, 100 kHz, 150 kHz, 200 kHz, 300 kHz, 400 kHz, 500 kHz, 1 MHz, or any frequency between.

In some aspects, the frequency of the alternating electric field is from about 150 kHz to about 300 kHz, from about 100 kHz to about 300 kHz from about 200 kHz to about 400 kHz, from about 250 kHz to about 350 kHz, and may be around 300 kHz.

In some aspects, the field strength of the alternating electric fields can be between 1 and 5 V/cm RMS. In some aspects, different field strengths can be used (e.g., between 0.1 and 10 V/cm). In some aspects, the field strength can be 1.75 V/cm RMS. In some embodiments the field strength is at least 1 V/cm. In some aspects, combinations of field strengths are applied, for example combining two or more frequencies at the same time, and/or applying two or more frequencies at different times.

In some aspects, the alternating electric field is applied at a frequency of between 250 kHz and 350 kHz. In some aspects, the alternating electric field is applied at a frequency between 50 and 190 kHz. In some aspects, the alternating electric field is applied at a frequency between 210 and 400 kHz. In some aspects, the alternating electric field has a field strength of at least 1 V/cm RMS. In some aspects, the alternating electric field has a frequency between 50 and 190 kHz. In some aspects, the alternating electric field has a frequency between 210 and 400 kHz. In some aspects, the alternating electric field has a field strength of at least 1 V/cm RMS. In some aspects, the alternating electric field has a field strength between 1 and 4 V/cm RMS.

In some aspects, the alternating electric fields can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric fields can be applied every day for a two hour duration.

In some aspects, the exposure may last for at least 6 hours, at least 12 hours, at least 24 hours, at least 36 hours, at least 48 hours, or at least 72 hours or more.

### G. Devices

Disclosed are methods of preventing or treating a virus infection in a subject comprising: applying an alternating electric field to a target site of the subject for a period of time, the alternating electric field having a frequency and field strength. In some aspects, the disclosed methods can be used for any virus that relies on an electrostatic interaction with a receptor. For example, the virus can be a coronavirus or lentivirus. Described herein are also devices that can be used to apply an alternating electric field to a target site of a subject.

Disclosed are methods of preventing or treating a coronavirus infection in a subject comprising: applying an alternating electric field to a target site of the subject for a period of time, the alternating electric field having a frequency and field strength. Described herein are also devices that can be used to apply an alternating electric field to a target site of a subject.

Disclosed herein are devices for use in the disclosed methods. For example, disclosed are devices for use in the disclosed methods of inhibiting a coronavirus from infecting or replicating in a cell. Disclosed are devices for use in the disclosed methods of reducing coronavirus copy number per cell. Disclosed are devices for use in the disclosed methods of treating a subject infected with coronavirus. Disclosed are devices for use in the disclosed methods of preventing the spread of coronavirus from a subject infected with a coronavirus to a non-infected subject.

In some aspects, the disclosed device can be an apparatus for electrotherapeutic treatment. Generally, the apparatus can be a portable, battery or power supply operated device that produces alternating electrical fields within the body by means of transducer arrays or other electrodes. The apparatus can comprise an electrical field generator and one or more electrode (e.g., transducer) arrays, each comprising a plurality of electrodes. The apparatus can be configured to generate tumor treating fields (TTFields) (e.g., at 150 kHz) via the electrical field generator and deliver the TTFields to an area of the body through the one or more electrode arrays. The electrical field generator can be a battery and/or power supply operated device.

The electrical field generator can comprise a processor in communication with a signal generator. The electrical field generator can comprise control software configured for controlling the performance of the processor and the signal generator. Although it can be within the electrical field generator, it is contemplated that the processor and/or control software can be provided separately from the electrical field generator, provided the processor is communicatively coupled to the signal generator and configured to execute the control software.

The signal generator can generate one or more electric signals in the shape of waveforms or trains of pulses. The signal generator can be configured to generate an alternating voltage waveform at frequencies in the range from about 50 KHz to about 1 MHz (preferably from about 100 KHz to about 300 KHz) (e.g., the TTFields). The voltages are such that the electrical field intensity in tissue to be treated is typically in the range of about 0.1 V/cm to about 10 V/cm.

One or more outputs of the electrical field generator can be coupled to one or more conductive leads that are attached at one end thereof to the signal generator. The opposite ends of the conductive leads are connected to the one or more electrode arrays that are activated by the electric signals (e.g., waveforms). The conductive leads can comprise standard isolated conductors with a flexible metal shield and can be grounded to prevent the spread of the electrical field generated by the conductive leads. The one or more outputs can be operated sequentially. Output parameters of the signal generator can comprise, for example, an intensity of the field, a frequency of the waves (e.g., treatment frequency), a maximum allowable temperature of the one or more electrode arrays, and/or combinations thereof. In some aspects, a temperature sensor can be associated with each electrode array. Once a temperature sensor measures a temperature above a threshold, current to the electrode array associated with said temperature sensor can be stopped until a second, lower threshold temperature is sensed. The output parameters can be set and/or determined by the control software in conjunction with the processor. After determining a desired (e.g., optimal) treatment frequency, the control software can cause the processor to send a control signal to the signal generator that causes the signal generator to output the desired treatment frequency to the one or more electrode arrays.

The one or more electrode arrays can be configured in a variety of shapes and positions so as to generate an electrical field of the desired configuration, direction and intensity at a target site (referred to herein also as a “target volume” or a “target region”) so as to focus treatment. Optionally, the one or more electrode arrays can be configured to deliver two perpendicular field directions through the volume of interest.

In some aspects, a first responder/healthcare professional can wear the disclosed device for use in one or more of the disclosed methods. For example, a first responder that is exposed to coronavirus can be treated with the electric fields within the device thus preventing any further possible spread of the virus.

### H. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example, disclosed are kits for imaging and/or treating. In some aspects, the kit can comprise devices and other equipment for applying alternating electrical fields to a subject.

Also disclosed are kits comprising a system or equipment for administering alternating electrical fields and one or more of the disclosed second therapeutics, such as, but not limited to, antiviral therapeutics.

### I. Embodiments

Embodiment 1: A method of inhibiting a coronavirus from infecting or replicating in a cell comprising: exposing the cell to an alternating electric field for a period of time, the alternating electric field having a frequency and field strength, wherein the frequency and field strength of the alternating electric field inhibits coronavirus infection or replication.

Embodiment 2: A method of reducing coronavirus copy number per cell comprising: exposing the cell to an alternating electric field for a period of time, the alternating electric field having a frequency and field strength, wherein the frequency and field strength of the alternating electric field reduces virus copy number in the cell.

Embodiment 3: A method of treating a subject infected with coronavirus comprising: applying an alternating electric field to a target site of the subject for a period of time, the alternating electric field having a frequency and field strength, wherein the target site comprises one or more coronavirus infected cells. In some aspects, embodiment 3 can be a medical use as shown here. Alternating electric fields for use in the treatment of a coronavirus infection comprising: applying alternating electric fields to a target site of a subject having a coronavirus infection for a period of time, the alternating electric fields having a frequency and field strength, wherein the target site comprises one or more coronavirus infected cells.

Embodiment 4; The method of any preceding embodiment, wherein the coronavirus is SARS-CoV-2.

Embodiment 5; The method of any preceding embodiment, wherein the alternating electric field is applied at a frequency of between 250 kHz and 350 kHz.

Embodiment 6; The method of any preceding embodiment, wherein the alternating electric field is applied at a frequency between 50 and 190 kHz.

Embodiment 7; The method of any preceding embodiment, wherein the alternating electric field is applied at a frequency between 210 and 400 kHz.

Embodiment 8; The method of any preceding embodiment, wherein the alternating electric field has a field strength of at least 1 V/cm RMS.

Embodiment 9; The method of any preceding embodiment, wherein the alternating electric field has a frequency between 50 and 190 kHz.

Embodiment 10; The method of any preceding embodiment, wherein the alternating electric field has a frequency between 210 and 400 kHz.

Embodiment 11; The method of any preceding embodiment, wherein the alternating electric field has a field strength of at least 1 V/cm RMS.

Embodiment 12; The method of any preceding embodiment, wherein the alternating electric field has a field strength between 1 and 4 V/cm RMS.

Embodiment 13; The method of any preceding embodiment, wherein the frequency of the alternating electric fields is 150 kHz.

Embodiment 14; The method of any preceding embodiment, wherein the parameters of the alternating electric fields is 150 kHz and 1.7 V/cm.

Embodiment 15; The method of any preceding embodiment, wherein the parameters of the alternating electric fields is about 150 kHz and about 1.5 V/cm.

Embodiment 16; The method of any preceding embodiment, wherein the target site of the subject is the lungs.

Embodiment 17; The method of any preceding embodiment, wherein the alternating electric field is applied multidirectionally.

Embodiment 18; The method of embodiment 17, wherein multidirectionally is at least two directions.

Embodiment 19; The method of any preceding embodiment, wherein cells not infected with a coronavirus are not damaged.

Embodiment 20; The method of any preceding embodiment, wherein cell viability is maintained.

Embodiment 21; The method of any preceding embodiment, wherein viral load in the subject is decreased and cell proliferation is unaffected.

Embodiment 22; The method of any preceding embodiment, wherein the viability of the cells at the target site is maintained and viral replication or viral infection is decreased.

Embodiment 23; A method of preventing the spread of coronavirus from a subject infected with a coronavirus to a non-infected subject comprising: applying an alternating electric field to a target site of the subject infected with a coronavirus for a period of time, the alternating electric field having a frequency and field strength, wherein the target site comprises one or more coronavirus infected cells.

Embodiment 24; The method of embodiment 23, wherein the alternating electric field results in the subject infected with coronavirus producing a defective virus, wherein the defective virus is less infectious than the wild type coronavirus.

Embodiment 25; A method of reducing replication of a coronavirus in a cell comprising: exposing the cell to an alternating electric field for a period of time, the alternating electric field having a frequency and field strength, wherein the frequency and field strength of the alternating electric field reduces virus copy number in the cell.

Embodiment 26; A method of preventing coronavirus infection in a subject comprising: applying an alternating electric field to a target site of the subject for a period of time, the alternating electric field having a frequency and field strength.

Embodiment 27; The method of embodiment 26, wherein the subject is not infected with coronavirus.

Embodiment 28; The method of embodiments 26 or 27, wherein the subject is at risk of being infected with coronavirus.

Embodiment 29; A method of treating a subject at risk for infection with coronavirus comprising: applying an alternating electric field to a target site of the subject for a period of time, the alternating electric field having a frequency and field strength.

Embodiment 30; A method of preventing a virus from getting close enough to a cell to infect the cell comprising: applying an alternating electric field to a target site of the subject for a period of time, the alternating electric field having a frequency and field strength.

Embodiment 31; A method of increasing fusion of a virus with a lysosome comprising: applying an alternating electric field to a target site of the subject for a period of time, the alternating electric field having a frequency and field strength, wherein the alternating electric field results in fusion of the virus with the lysosome.

Embodiment 32: The method of embodiment 1 or 2, wherein the cell is in a subject.

Embodiment 33: The method of any preceding embodiment, wherein the alternating electric field is applied at a frequency of between 50 kHz and 1 MHz.

### EXAMPLES

### A. Example 1

Coronavirus disease 2019 (COVID-19) is an infectious disease caused by severe acute respiratory syndrome coronavirus 2 (SARS-CoV-2). It was first identified in December 2019 in Wuhan, Hubei, China, and has resulted in an ongoing pandemic. As of 19th of August 2020, close to 22 million cases have been reported across 188 countries and territories, resulting in more than 775,000 deaths (www.who.int/emergencies/diseases/novel-coronavirus-2019). Management involves the treatment of symptoms, supportive care, isolation, and experimental measures. Supportive care, may include fluid therapy, oxygen support, and supporting other affected vital organs. There is an urgent unmet need to develop new therapies for the treatment of COVID-19.

Tumor Treating Fields (TTFields) are low intensity (1-5 V/cm), intermediate frequency (100-500 kHz) alternating electric fields that were shown to disrupt cancer cell division. TTFields application generate an electric fields inside and around cells thus inducing forces on dipoles and polarizable objects. TTFields application was shown to disrupt the organization of cellular structural elements whose assembly relies upon electrostatic interaction between subunits with high electrical dipoles. In addition, TTFields application was demonstrated to induce replication stress and collapse of the replication fork. Cell death induced by TTFields had the characteristics of immunogenic cell death thus priming the immune system. TTFields application in the clinic demonstrated increase overall survival in some of the most aggressive types of cancers, with minimum adverse events, leading to FDA approval of the modality for the treatment of newly diagnosed glioblastoma, recurrent glioblastoma and malignant pleural mesothelioma. Recent studies demonstrated that TTFields application can inhibit lentivirus replication and can reduce the percentile of infected cells (unpublished), yet, there is no data on the potential effect of TTFields on COVID-19.

The aim of this work was to study TTFields effect on the coronavirus in the in vitro settings.

1. Summary

Tumor Treating Fields (TTFields) are low intensity (1-5 V/cm), intermediate frequency (100-500 kHz) alternating electric fields that were shown to disrupt cancer cell division. TTFields application generate an electric fields inside and around cells thus inducing forces on dipoles and polarizable objects. TTFields application in the clinic demonstrated increase overall survival in some of the most aggressive type of cancers, with minimum adverse events, leading to FDA approval of the modality for the treatment of newly diagnosed glioblastoma, recurrent glioblastoma and malignant pleural mesothelioma (MPM). Recent studies demonstrated that TTFields application can inhibit lentivirus replication and can reduce the percentile of infected cells.

Human coronavirus HCoV-229E was used to study the effect of two directional TTFields (150 kHz, 1.7 V/cm) on the number of viruses produced by MRC5 cells infected before, during or after treatment. The number of surviving cells, virus per ml, virus per cell and RQ (virus particles normalized to host housekeeping gene) were determined at the end of treatment.

Results demonstrate that in cells treated with TTFields for 48 hours during the 6 hours infection phase and the 42 hours replication phase, there was a significant reduction in the virus copy number per cell (32.4% virus copies per cells as compared to the control, p<0.0001) and the virus copies per the housekeeping gene (RQ=0.284 in the TTFields arm vs 1.561 in the control arm, p<0.0001). Similar trends were observed when TTFields were applied for 24 hours during the 6 hours infection phase and the 18 hours replication phase, yet the results were not statistically significant. In cultures treated for 48 hours, virus copy number in the supernatant was also reduced (297,345 copies) as compared to the control (1,411,880 copies) (p<0.0001). In infected cultures treated with TTFields for 24 or 48 hours, significantly more cells survived the insult of the HCoV-229E infection as compared to untreated cultures.

Taken together, the results of this study provide evidence that TTFields can inhibit coronavirus infection and replication. Of note, TTFields parameters used in this study (150 kHz, 1.7 V/cm) are similar both in frequency and intensities to the treatment delivered by the Optune Lua system (Novocure Ltd) which received FDA approval for the treatment of malignant pleural mesothelioma (MPM).

2. Materials and Methods

i. Cell Line and Virus

MRC5 cells (ATCC, CCL-171™) and HCoV-229E (ATCC, VR740™) were provided by Dr. Michal Mendelbaum of Sheba medical center. The cells were grown in Eagle's Minimum Essential Medium (EMEM) (ATCC, 30-2003™) supplemented with 10% fetal bovine serum (FBS) (Biological Industries, 04-007-1A).

ii. Virus Infection and Proliferation

MRC5 cells were seeded on glass cover slips (22 mm diameter) at a density of 1.5×10⁵ cells/cover slip. After a 24-hour culture, the incubator temperature was changed from 37° C. to 35° C. and the cells were transferred into inovitro dishes containing 2 ml EMEM supplemented with 2% FBS. The cells were infected with the HCoV-229E virus at 0.01% concentration over 6 hours. Following infection, the cells were washed with PBS and maintained for additional 18 hours or 42 hours in EMEM supplemented with 2% FBS. TTFields were applied either during the infection and proliferation phases. At 24 hours or 48 hours after infection, the growth media were collected, cells were trypsinized, resuspended and counted using Scepter™ 2.0 Cell Counter (Merck, Millipore). After resuspension the cells were washed with PBS. The supernatants and pellets were frozen at −80° C. and transferred to Sheba for viral genome extraction and qRT-PCR.

iii. TTFields Application

TTFields (150 kHz, 1.7 V/cm RMS) were applied using the Inovitro™ system (Novocure Ltd) as described with the following amendments: the inovitro plates were covered with parafilm and the media were not replaced during cell culture.

iv. Statistical Analysis

Each experiment was repeated three times. Data from all repeats were pooled for the analysis. Data are presented as means±SD. Statistical significance was analyzed by the Kruskal-Wallis Test.

3. Results

Following preliminary testing, 0.01% virus/cells infection ratio was used in all experiments. TTFields ((150 kHz, 1.7 V/cm) were applied during the 6 hours of infection and 18 hours or 42 hours of virus replication for a total duration of 24 or 48 hours. Three independent repeats were conducted for each group. Due to the variability between repeats, the following parameters were normalized to the average control of each repeat: cell counts, virus copies/cell and virus copies in supernatant (SN).

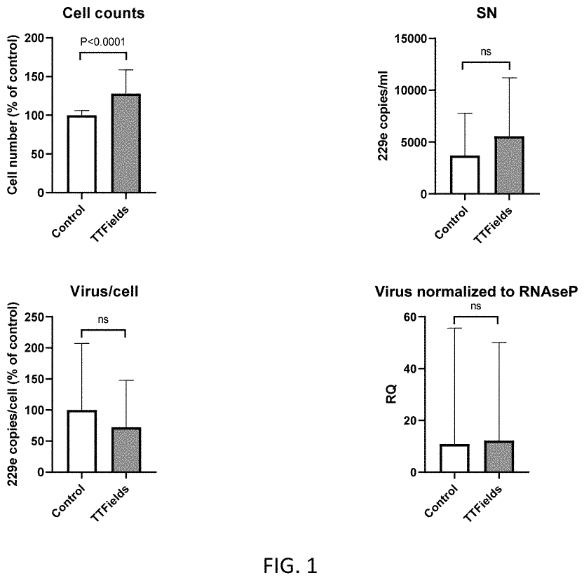

There were significantly more cells at treatment end in infected cultures treated with TTFields for 24 hours (p<0.0001) as compared to the untreated cultures. There was no significant difference between the virus copies in the supernatant (SN) and in the number of the virus copy number compared to the copies of the housekeeping gene (RQ), as compared to the control. TTFields application for 24 hours led to some reduction in the virus copy number per cell yet the results did not reach statistical significance (See FIG. 1).

As TTFields efficacy is known to be dependent on treatment duration, in the next set of experiments, TTFields application duration was extended to 48 hours.

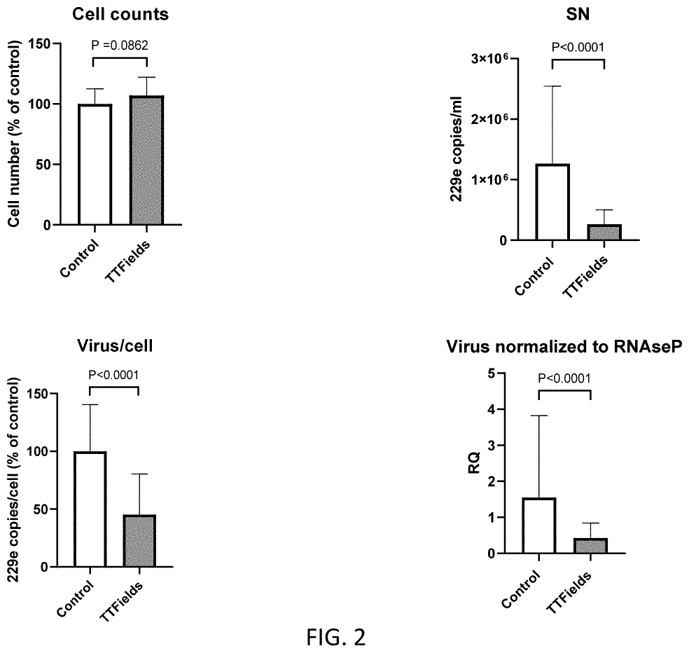

There were significantly more cells at treatment end in infected cultures treated with TTFields for 24 hours (p<0.01) as compared to the untreated cultures. TTFields application for 48 hours led to a significant reduction in the virus copies in the supernatant (SN) as compared to the control (p<0.01) and to significant reduction in the virus copy number per cell (p<0.0001) and in the number of the virus copy number compared to the copies of the housekeeping gene (RQ) (p<0.0001) (See FIG. 2).

4. Conclusions

As disclosed herein, TTFields were applied to MRC5 cell cultures infected with the coronavirus 229E. The frequency (150 kHz) and the intensities (1.7 V/cm) used are comparable to the ones being applied by the Optune Lua device which received FDA approval for the treatment of malignant pleural mesothelioma following the successful STELLAR clinical study which demonstrated treatment efficacy and safety.

The virus copy number per cell was reduced following 24 hours treatment with TTFields. TTFields efficacy is known to be dependent on treatment duration and therefore in the second set of experiments, TTFields were applied for 48 hours. Indeed, extending treatment duration resulted in significant reductions in the virus copy numbers per cell, per the housekeeping gene and in the supernatant.

TTFields application for 48 hours at 150 kHz and 1.7 V/cm was demonstrated to effectively kill dividing cancer cells. Yet, in this study, the number of infected cells treated with TTFields was higher as compared to infected cultures not treated with TTFields.

Taken together the results of this study provide evidence for the use of TTFields as a treatment against COVID-19 using the currently available Optune Lua system.

### B. Example 2

The aim of this study was to test the feasibility of using TTFields for treating coronavirus. Additional experiments were performed on cells to determine the effect of TTFields on virus copy number per cell of infected cells as well as on cells not infected with virus.

Cells were infected with human coronavirus 229E and then exposed to TTFields as well as cultures not exposed to TTFields. Cells not infected with human coronavirus 229E were also exposed to TTFields.

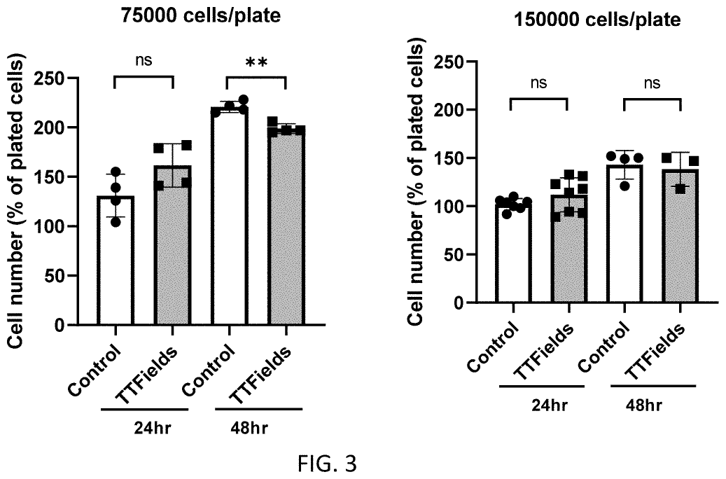

The number of cells was not significantly different in culture treated with TTFields vs. cultures which were not treated with TTFields (FIG. 3). Only when comparing cultures which were infected with 229E was there a survival benefit of MRC5 cells following treatment with TTFields, compared to infected cells not treated with TTFields.

As non-infected cells grow faster with TTFields than without TTFields, it may be valuable to present the same results after normalizing to this effect as well. Results were normalized to the number of cells in the control cultures.

The number of the virus detector copies from cell pellets was normalized per the housekeeping gene (RNAseP). RQ—relative quantification strategy for quantitative RT-PCR data analysis. This method has been used to calculate relative 229E virus gene expression levels between samples from cell pellets with or w/o TTFields treatment. RQ method directly uses the threshold cycles (CTs) generated by the qPCR system for calculation.

The report shows the accumulated/normalized data from all 3 experiments. Results demonstrate that in cells treated with TTFields, there was a significant reduction in the virus copy number per cell (FIG. 2). Similar trends were observed in all three experiments performed.

The cells are treated with two TTFields directions. The human device always utilizes bi-directional TTFields, as well as all standard in vitro and in vivo studies.

The trend was identical during the 24 hour application. Since TTFields are already known to affect processes which are related to DNA replication and integration, it is believed that the effects seen through the reported experiments were related to the intracellular activity of the virus and the cumulative effect became statistically significant following the prolonged application of the fields, as seen in cancer models, too.

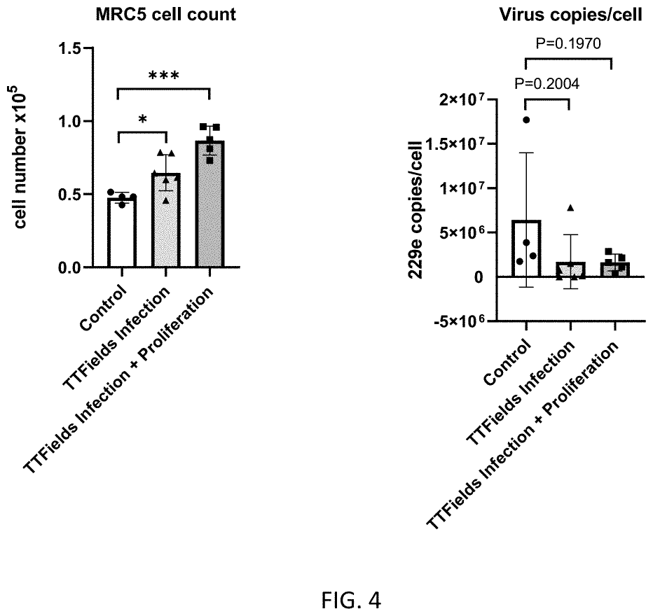

A reduction in viral load was seen after 24 h in both supernatant (SN) and cells that did not significantly change whether TTFields were applied during infection only (6 h) or during the entire 24 h (FIG. 4).

TTFields application for 48 hours at 150 kHz and ˜1.5 V/cm resulted in significant reductions in the virus copy numbers per cell, per the housekeeping gene and in the supernatant. The results of this study provide evidence for the use of TTFields as a treatment against COVID-19 using the currently available Optune Luna system.

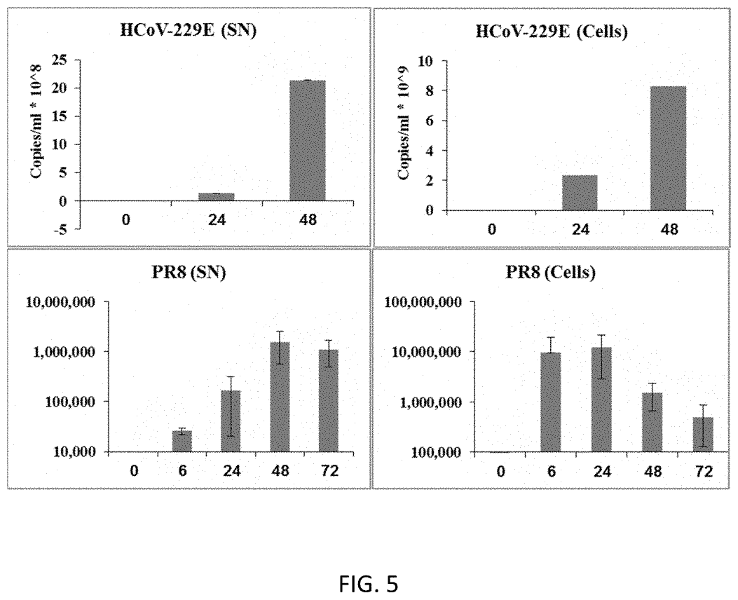

FIG. 5 shows human coronavirus 229E in MRC5 cells (human fibroblasts from lungs) and mouse-adapted influenza virus PR8 in A549 cells (human lung carcinoma). After 24 hours the 229E virus shows increase in virus copies/ml of cell lysate, and after 48 hours the increase in the media of the cells. For PR8, the virus copies are detectable already after 6 hours.

FIG. 3 shows the effect of TTFields on MRC5 cells. Cell number did not significantly change between the control and TTFields treated MRC5 cells.

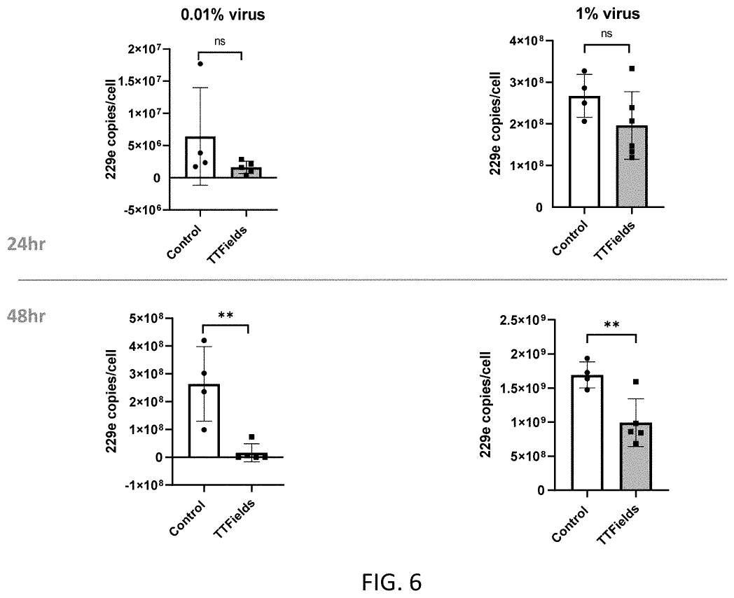

FIG. 6 shows an example of an effect of TTFields on 229E depending on viral concentration. Viral copy number/cell was measured at 24 hr and 48 hr after TTFields. Cells were infected with either 0.01% virus or 1% virus. After 48 hrs, the viral copy number/cell significantly decreased with treatment of TTFields regardless of virus concentration. It can be noted that TTFields had a greater effect on the lower virus to cell ratio and since chronic infection can occur with lower viral loads, TTFields can be used for chronic infection.

FIG. 4 shows TTFields during infection or infection and replication phase. 0.01% virus was used to infect the cells. TTFields infection occurred for 6 hours. TTFields infection plus proliferation occurred for 18 hours. Although the amount of cells increased in the TTFields treated plates was larger than in control, the virus copy number/cell decreased in the TTFields treated cells. Healthy (non-infected) cells are not affected by TTFields. Lots of therapeutics can damage healthy cells along with the “bad” cells or unhealthy/infected cells. TTFields show minimal damage to healthy cells thus having an added benefit of this safety factor when using as a treatment for coronavirus.

Following preliminary testing, 0.01% virus/cells infection ratio was used in both experiments (FIG. 1 and FIG. 2). TTFields ((150 kHz, 1.5 V/cm) were applied during the 6 hours of infection and 18 hours or 42 hours of virus replication for a total duration of 24 (FIG. 1) or 48 (FIG. 2) hours. Four independent repeats were conducted for each group. Due to the variability between repeats, the following parameters were normalized to the average control of each repeat: cell counts, virus copies/cell and virus copies in supernatant (SN).

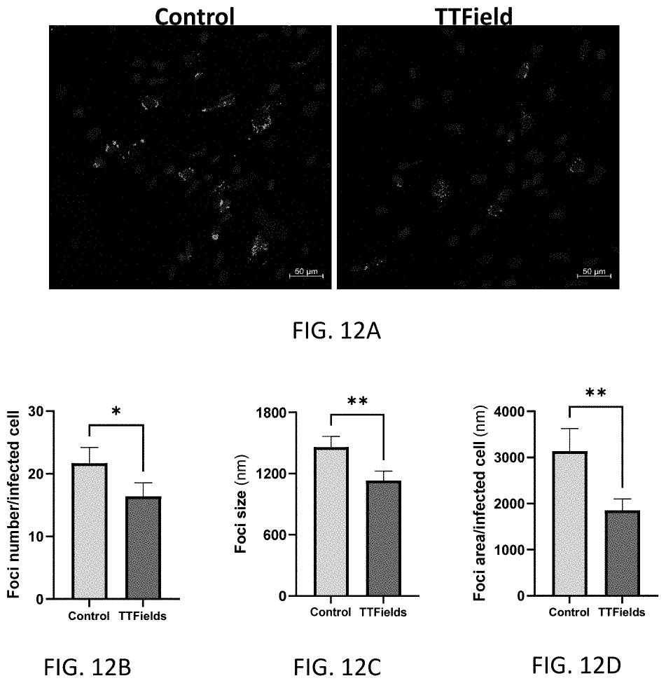

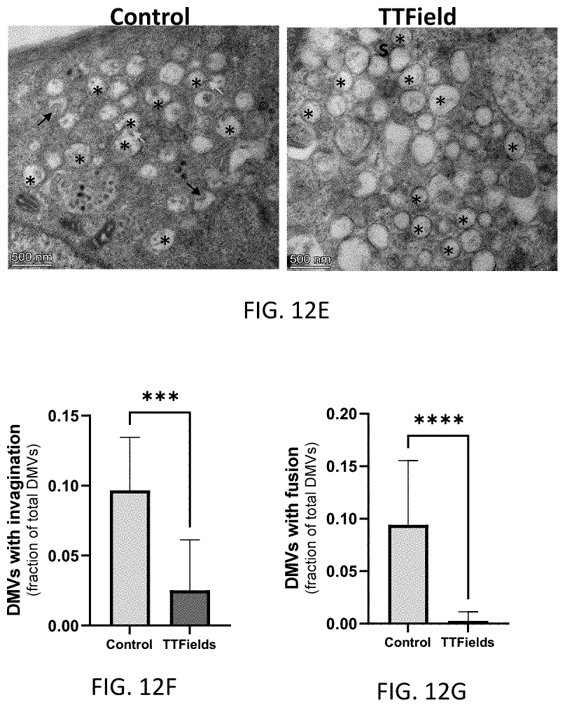

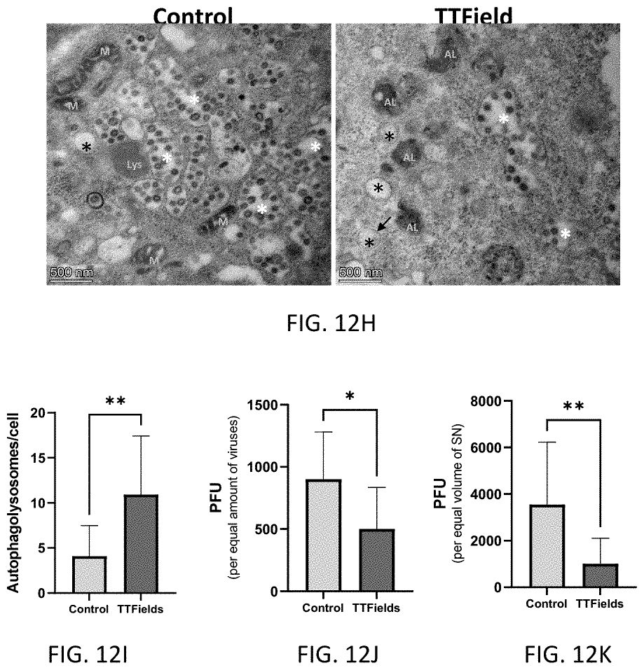

There were significantly more cells at treatment end in infected cultures treated with TTFields for 24 hours (p<0.0001) as compared to the untreated cultures. There was no significant difference between the virus copies in the supernatant (SN) and in the number of the virus copy number compared to the copies of the housekeeping gene (RQ), as compared to the control. TTFields application for 24 hours led to some reduction in the virus copy number per cell yet the results did not reach statistical significance (See FIG. 12).

In the next set of experiments, TTFields application duration was extended to 48 hours. There were more cells at treatment end in infected cultures treated with TTFields for 48 hours (p=0.0862) as compared to the untreated cultures. TTFields application for 48 hours led to a significant reduction in the virus copies in the supernatant (SN) as compared to the control (p<0.0001) and to significant reduction in the virus copy number per cell (p<0.0001) and in the number of the virus copy number compared to the copies of the housekeeping gene (RQ) (p<0.0001) (See FIG. 2).

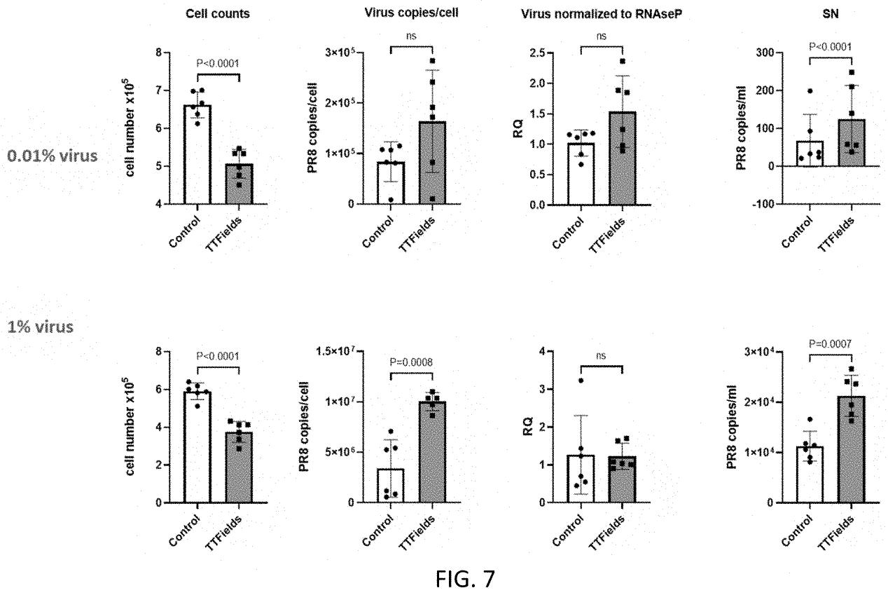

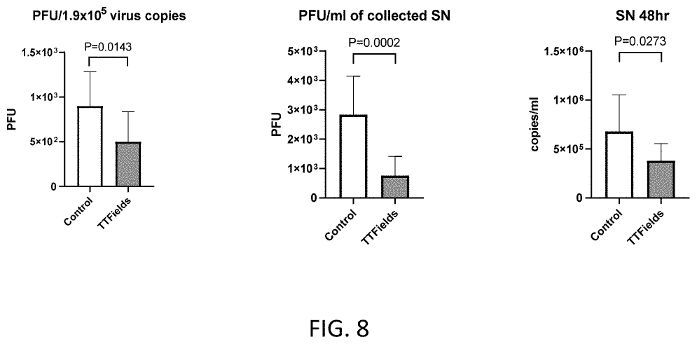

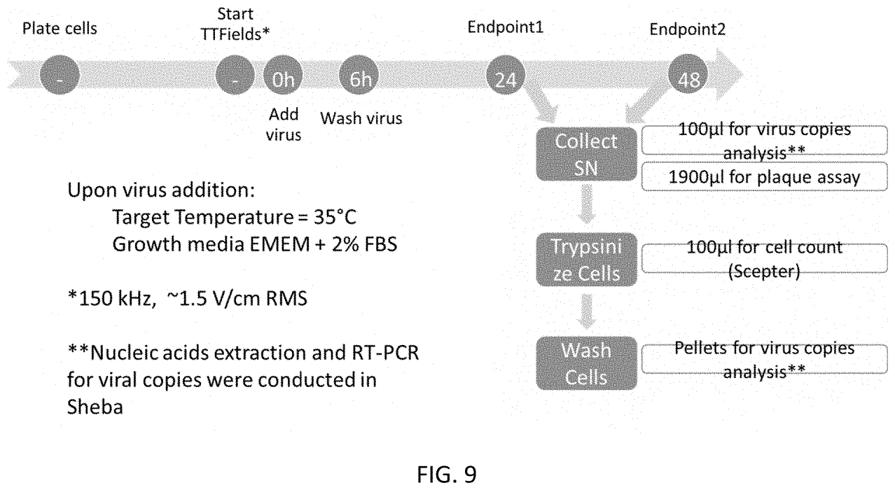

The virus copy number per cell was slightly reduced following 24 hours treatment with TTFields yet the results did not reach statistical significance. TTFields efficacy is known to be dependent on treatment duration and therefore in the second set of experiments, TTFields were applied for 48 hours. Indeed, extending treatment duration resulted in significant reductions in the virus copy numbers per cell, per the housekeeping gene and in SN. Moreover, TTFields treatment led also to a significant reduction in the concentrations of replication competent lytic virions (PFU) (see FIG. 8).

1.5×10⁵ MRC5 cells were infected with 229E virus and treated with TTFields during infection or during infection and replication phases. After 24 hours there was approximately 40% reduction in the cell count of cells that were treated during two phases, approximately 55% reduction in the cell count of cells that were treated during only infection and approximately 75% reduction in the cell count of Control cells (FIG. 4 Left). The virus copy number per cell was slightly reduced following 24 hours treatment with TTFields in both treatments (infection only or infection+replications).

FIG. 7 shows an example of an effect of 48 hr TTFields treatment on PR8 virus in A549 cells. Contrary to the 229e virus, the PR8 virus experiments showed a decrease in cell number when comparing the TTFields treated cells to the control. There was also an increase in virus copy number/cell in the TTFields treated cells compared to the control. Thus, either the PR8 virus did not behave the same as the 229e virus in response to TTFields or there is a different effect of TTFields on interaction between virus and cancer or healthy cells.

### C. Example 3

1. Plaque Assay

Plaque assays can serve as a quantitative method of measuring infectious coronavirus by quantifying the plaques formed in cell culture upon infection with serial dilutions of a virus specimen. As such, plaque assays remain the gold standard in quantifying concentrations of replication competent lytic virions. This Example describes a plaque assay performed to quantify 229E in part of the supernatants which were collected from coronavirus infected MRC5 cells after 48 hr treatments.

i. Reagents and Solutions

a. Infection Media:

Eagle's Minimum Essential Medium (EMEM) (ATCC, 30-2003™) supplemented with 2% fetal bovine serum (FBS) (Biological Industries, 04-007-1A).

b. Overlay Diluent:

DMEM×2—500 ml

Pen-Strep—10 ml

Glutamine—10 ml

Pyruvate—10 ml

FBS—20 ml

c. Carboxymethylcellulose (CMC), 3% (w/v)

6 g carboxymethylcellulose, medium viscosity (solid; Sigma-Aldrich #C4888-500G),

200 ml distilled water

Sterilized by autoclaving at 121° C. for 15 min

Stored at room temperature up to 2 months

d. Crystal Violet, 1% (w/v)

1 g crystal violet

20 ml absolute ethanol

80 ml distilled water

Filter sterilize

Stored at room temperature

2. Methods

i. Cell Culture (Day 0)

Seed plates: Cells were trypsinized. 12-well plates were seeded with 2×10⁵ cells per well by adding 1 ml of the cell suspension to each well. Incubate at 37° C. in a 5% CO2 incubator overnight to achieve 100% confluence the following day. Cells were visualized the following day using a light microscope. Cells that reached 95% to 100% confluence were infected.

ii. Specimen Dilution, Infection of Cells, and Primary Overlay (Day 1)

Specimen dilution: For each specimen to be titrated, 2 ml microcentrifuge tubes were labeled as follows:

(1.) Negative Control (infection media only); (2.) 190571 virus copies in 1 ml infection media; and (3.) 10⁻¹ of No 2 (add 100 μl of No 2 to 900 μl media)

Infection: Medium from cell monolayers were aspirated. Cells were washed with 1 ml of PBS. 900 μl of infection medium were added to each well. Virus specimens were diluted 10-fold by transferring 100 μl of each specimen to the appropriate well. Cells were incubated at 35° C. in a 5% CO2 incubator for 2 hr.

Preparation of Liquid overlay media (LOM): Approximately 10 min before incubation is over overlay diluent was incubated at 44° C. in a water bath. LOM was prepared by adding equal amounts (1:1) of overlay diluent and 3% CMC (liquid matrix).

Cells were washed twice with 1 ml of PBS. 1 ml of LOM was added to each well of the plates. Cells were incubated at 35° C. in a 5% CO2 incubator for 4 days. During this incubation, movement of the plates was minimized.

iii. Fixation, Staining, Enumeration of Plaques, and Titer Calculation (Day 5)

a. Fixation:

LOM was aspirated from each well and discard in a waste bottle. Cells were gently washed twice with PBS×1 and fill wells with ice cool ethanol absolute and the incubated at −20° C. for 15 min.

b. Staining:

Fixative was aspirated from wells and waste discarded. 300 μl of 1% CV was added to each well and incubate at room temperature for 5 minutes.

c. Wash:

Cells were washed 3-4 times with distilled water and blotted dry. At this point, plates can be surface-decontaminated with an appropriate disinfectant can be stored at 4° C. for up to 1 month.

d. Enumerate Plaques:

Plaques will appear as clear circles on a purple monolayer of cells. The negative control should have a uniform monolayer, which can be used as a reference. The number of plaques observed per well at each virus dilution were recorded.

e. Titer Calculation:

PFU/[ml infection]=PFU per well×dilution factor

PFU/[ml collected SN]=PFU×1000/infection volume of SN in 1 ml infection media.

iv. Plaque Assay

MRC5 cells were seeded in 12 well plates at a density of 2×10⁵ cells/well. After a 24-hour culture, the incubator temperature was changed from 37° C. to 35° C. and the medium was replaced with the new 1 ml EMEM supplemented with 2% FBS. The cells were infected with the 1.9×10⁵ virus copies over 2 hours. After infection, the cells were washed with PBS and covered with medium supplemented with 2% FBS and 1.5% CMC. Four days after infection the cells were fixed with ice-cold ethanol absolute for 15 min and stained with 1% Cristal Violet for 5 min at room temperature. The plaque forming units (PFU) were counted and the PFU/ml SN was calculated (PFU×1000/infection volume of SN in 1 ml infection media).

3. Results

This assay was used for quantification of 229E in part of the supernatants which were collected from coronavirus infected MRC5 cells after 48 hr treatments. TTFields application for 48 hr led to a significant reduction in the PFU per an equal amount of virus copies in each treatment (P=0.0143) and to an additional reduction in PFU per milliliter of the supernatants (P=0.0002) (See FIG. 8. The stronger reduction in the PFU per ml of supernatant can be explained by the mutual effect of reduction in the number of competent viruses and reduction in the virus copies total.

### D. Example 4—Clinical Trial Study

Disclosed is a pilot, randomized, open-label study of Optune-Lua® (TTFileds, 150 Hz) compared with best standard of care in hospitalized patients with COVID-19. The objective of this study is to evaluate the effectiveness and safety of TTFields concomitant with best standard of care (SOC) compared to best SOC for treating patients with COVID-19.

Data provided throughout this application shows that TTFields can significantly reduce coronavirus infection and replication in vitro. The frequency (150 kHz) and the intensities (1.7 V/cm) used in this model are comparable to the ones being applied by the Optune Lua (NovoTTF-100L) device which received FDA approval for the treatment of MPM following the successful STELLAR clinical study which demonstrated treatment efficacy and safety.

Taken together, as a novel, safe and regional therapy with demonstrated preclinical efficacy, TTFields (150 kHz) can be delivered using the NovoTTF-100L system and have the potential to become an effective treatment for COVID-19.

This Example can show the addition of TTFields, delivered using the NovoTTF-100L System to the thorax, to SOC as treatment for COVID-19 disease, significantly improves the clinical outcome of patients, compared to the SOC treatment alone.

In some aspects, this study can evaluate if TTFields at 150 kHz to the thorax with SOC in the treatment of hospitalized patients with COVID-19 shortens the time to recovery, compared to SOC treatment alone.

In some aspects, this study can evaluate if TTFields at 150 kHz to the thorax with SOC in the treatment of hospitalized patients with COVID-19 improves the clinical status of the patients at day 8, 15, 22 and 29, compared to SOC treatment alone.

In some aspects, this study can evaluate if TTFields at 150 kHz to the thorax with SOC in the treatment of hospitalized patients with COVID-19 shorten the duration of hospitalization, compared to SOC treatment alone.

In some aspects, this study can evaluate if TTFields at 150 kHz to the thorax with SOC in the treatment of hospitalized patients with COVID-19 decrease all cause mortality rate, compared to SOC treatment alone.

In some aspects, this study can evaluate if TTFields at 150 kHz to the thorax with SOC in the treatment of hospitalized patients with COVID-19 decrease the incidence of ICU admission, compared to SOC treatment alone.

In some aspects, this study can evaluate if TTFields at 150 kHz to the thorax with SOC in the treatment of hospitalized patients with COVID-19 decrease the duration of ICU stay, compared to SOC treatment alone.

In some aspects, this study can evaluate if TTFields at 150 kHz to the thorax with SOC in the treatment of hospitalized patients with COVID-19 decrease the incidence non-invasive ventilation or high-flow oxygen use, compared to SOC treatment alone.

In some aspects, this study can evaluate if TTFields at 150 kHz to the thorax with SOC in the treatment of hospitalized patients with COVID-19 shorten the duration of non-invasive ventilation or high-flow oxygen use, compared to SOC treatment alone.

In some aspects, this study can evaluate if TTFields at 150 kHz to the thorax with SOC in the treatment of hospitalized patients with COVID-19 decrease the incidence of invasive ventilation, compared to SOC treatment alone.

In some aspects, this study can evaluate if TTFields at 150 kHz to the thorax with SOC in the treatment of hospitalized patients with COVID-19 shorten the duration of invasive ventilation, compared to SOC treatment alone.

In some aspects, this study can evaluate if TTFields at 150 kHz to the thorax with SOC in the treatment of hospitalized patients with COVID-19 decrease the incidence of ECMO, compared to SOC treatment alone.

In some aspects, this study can evaluate if TTFields at 150 kHz to the thorax with SOC in the treatment of hospitalized patients with COVID-19 shorten the duration of ECMO, compared to SOC treatment alone.

In some aspects, this study can evaluate if TTFields at 150 kHz to the thorax with SOC in the treatment of hospitalized patients with COVID-19 decrease the incidence of return to hospital, compared to SOC treatment alone.

In some aspects, this study can evaluate if TTFields at 150 kHz to the thorax with SOC in the treatment of hospitalized patients with COVID-19 increase the saturation levels and stability, compared to SOC treatment alone.

In some aspects, this study can evaluate if TTFields at 150 kHz to the thorax with SOC in the treatment of hospitalized patients with COVID-19 shorten the duration and level of body temperature, compared to SOC treatment alone.

In some aspects, this study can evaluate if TTFields at 150 kHz to the thorax with SOC in the treatment of hospitalized patients with COVID-19 shorten the duration of supplemental oxygenation, compared to SOC treatment alone.

In some aspects, this study can evaluate if TTFields at 150 kHz to the thorax with SOC in the treatment of hospitalized patients with COVID-19 decrease the patient inflammatory status, compared to SOC treatment alone.

In some aspects, this study can evaluate if TTFields at 150 kHz to the thorax with SOC in the treatment of hospitalized patients with COVID-19 improves the patient lung radiological assessment, compared to SOC treatment alone.

In some aspects, this study can evaluate if TTFields at 150 kHz to the thorax with SOC in the treatment of hospitalized patients with COVID-19 is safe, compared to SOC treatment alone.

1. Study Treatments

All subjects in the study regardless of study arm receive the best SOC treatment for COVID-19 disease.

i. The Device

The current study can use the Optune Lua® System. This NovoTTF-100L System is an investigational medical device delivering 150 kHz TTFields to the thorax for the treatment of patients at the age of 18 years or older with COVID-19. It can be used for at least 18 hours per day on a monthly average and exclusively by patients in a clinical study.

The device is a portable, battery operated system which delivers TTFields at 150 kHz to the thorax by means of insulated Transducer Arrays. The NovoTTF-100L produces electric forces intended to attenuate SARS-CoV-2 infection and replication.

ii. Applying TTFields Using the Device

Treatment planning: Transducer Array Layout can be determined based on the Clinical Practice Guidelines: Optimizing the transducer array layout in TTFields-treated patients (thoracic infectious disease).

Treatment initiation: NovoTTF-100L treatment can be initiated by the investigator within 24 hours following hospitalization. Subjects with hair where transducer arrays are planned to be applied can be required to shave it prior to transducer array placement. Transducer array placement can be performed based on the Transducer Array Layout map chosen prior to treatment initiation, avoiding areas of skin damage such as wounds.

The NovoTTF-100L System can be programmed to deliver 150 kHz TTFields to the thorax in two sequential, perpendicular field directions at a maximal intensity of 1414 mA (RMS).

Transducer array replacement: Subjects can replace the transducer arrays twice to three times per week with the help of a caregiver. At each transducer array replacement the subject's skin can be re-shaved if needed, and treated according to the guidelines set out below.

iii. Duration

TTFields at 150 kHz to the thorax can be continuous for at least 18 hours a day on average. Subjects can take breaks for personal needs (e.g. showering, transducer array exchange) as long as the average treatment remains 18 hours per day (monthly average). TTFields can continue for 29 days, or until the subject is not hospitalized and with no limitations on activities, death, unacceptable adverse event(s), intercurrent illness that prevents further administration of treatment, investigator's decision to withdraw the subject, subject withdraws consent, pregnancy of the subject, noncompliance with study treatment or procedure requirements, or administrative reasons.

2. Study Procedures and Schedule

i. Study Specific Procedures

The following data can be collected in the study.

Clinical status score: for each study day, the clinical status can be recorded on an 8-point ordinal scale as follows: Day 1—The clinical assessment at the time of randomization; Day 2—The most severe assessment occurring at any time between randomization and midnight the day of randomization; Day 3 and after—The most severe assessment occurring from midnight to midnight (00:00 to 23:59) of the prior day (e.g., the value recorded on Day 3 can be the most severe outcome that occurred on Day 2).

The clinical status scale can be defined as follows: Not hospitalized, no limitations on activities; Not hospitalized, limitation on activities; Hospitalized, not requiring supplemental oxygen—no longer requires ongoing medical care; Hospitalized, not requiring supplemental oxygen-requiring ongoing medical care (COVID-19 related or otherwise; Hospitalized, requiring supplemental oxygen; Hospitalized, on non-invasive ventilation or high flow oxygen devices; Hospitalized, on invasive mechanical ventilation or ECMO; or Death.

Medical history can include any clinically-significant history of the patient, focusing on the past 5 years and any other important history beyond 5 years. History can be obtained from medical records complemented by an interview with the patient. If medical records are unavailable, history can be obtained by interviewing the patient. Medical history includes smoking and significant co-morbidities.

Concomitant medications include both prescription and over-the-counter medications taken by the patient throughout the study period, including dose, frequency, indication, start and stop dates.

COVID-19 disease history includes the day of onset of COVID-19 symptoms and signs, including diagnosis date, date and result of previous SARS-CoV-2 RT-PCR tests, all past treatments.

Patient demographics can also be included.

Physical examinations can also be performed. Physical examinations can include heart rate, blood pressure, respiration rate, body temperature, weight, height, blood saturation level (SpO2). Examination and review of the following systems can be performed: head and neck, cardiac, pulmonary, abdominal, extremities, skin, neurological.

CT/X-Ray scans can also be performed and include, but are not limited to, thoracic scan, including the collection of complete data required for the assessment of COVID-19 lungs radiological status.

ii. Clinical Laboratory Evaluations

Blood tests can also be performed. Complete blood count and differential can include: hemoglobin, hematocrit, MCV, RBC, WBC, neutrophil count, eosinophil count, basophil count, lymphocyte count, monocyte count, platelet count. Chemistry can include: sodium, potassium, urea/BUN, creatinine, glucose, LDH, AST, ALT, albumin, bilirubin. Coagulation can include: PTT/aPTT, PT/INR. Inflammatory blood markers can include: CRP, D-dimer and ferritin. Pregnancy tests can be performed using serum beta-hCG testing.

RT-PCR SARS-CoV-2 OP swab test and RT-PCR SARS-CoV-2 blood test can also be performed.

iii. Screening

The following can be performed within 24 hours following hospitalization: Clinical status based on the 8-point ordinal scale; COVID-19 disease history (including review of previous SARS-CoV-2 test results); RT-PCR SARS-CoV-2 OP swab test; RT-PCR SARS-CoV-2 blood test; CT/X-Ray scan of the chest; Demographics and Medical history; Concomitant medications recording; Physical examination (including vital signs and SpO2 levels); Serum pregnancy test (if applicable); Complete blood count including differential; Serum chemistry panel; Coagulation tests; Inflammatory blood markers test.

iv. Randomization

Patients can be centrally randomized using an IxRS system at a 1:1 ratio to 2 treatment arms prior to treatment start: Treatment arm I: Patients receive TTFields at 150 kHz to the thorax using the NovoTTF-100L System together with SOC and Treatment arm II: Patients receive SOC alone.

3. Schedule of Events

An example of a schedule of events is shown in Table 1 below.

TABLE 1

| | | | | | POST- |
|---|---|---|---|---|---|
| | | IN HOSPITAL | | POST- | TREATMENT |
| | | FOLLOW UP | | DISCHARGE | TERMINATION |
| | SCREENING/ | | T = On | FOLLOW UP | VISIT |
| | BASELINE | | days 3, 5, | T = On days | T = 30-37 days |
| | T = (−24)-0 | T = Every | 8, 11, 15 | 8, 15, 22 and | following |
| | hours (baseline | day until | and 29 until | 29 following | treatment |
| | evaluation) | discharge | discharge | discharge | termination |
| Signed ICF | X | | | | |
| Demographics and Medical | X | | | | |
| history | | | | | |
| Concomitant medications | X | X | | X | X |
| recording | | | | | |
| Clinical status | X | X | | X | X |
| (based on the 8- | | | | | |
| point ordinal scale) | | | | | |
| COVID-19 disease history | X | | | | |
| Physical | X | | | | |
| examination | | | | | |
| (including vital signs | | | | | |
| and SpO2 levels) | | | | | |
| Targeted physical | | X | | X | |
| examination | | | | | |
| focused on lung | | | | | |
| auscultation | | | | | |
| (including vital signs | | | | | |
| and SpO2 levels) | | | | | |
| CT/X-Ray scan of the chest | X | | | | |
| RT-PCR SARS- | X | | X | | |
| CoV-2 blood test | | | | | |
| RT-PCR SARS- | X | | X | X⁴ | |
| CoV-2 OP swab test | | | | | |
| Serum pregnancy test | X¹ | | | | |
| (if applicable) | | | | | |
| Complete blood | X | | X | | |
| count including | | | | | |
| differential | | | | | |
| Serum chemistry panel | X | | X | | |
| Coagulation tests | X | | X⁵ | | |
| Inflammatory markers test | X² | | X² | | |
| Adverse event and | | | X | X | X |
| device deficiency | | | | | |
| collection and recording | | | | | |
| NovoTTF-100L device | | | X³ | X³ | |
| usage assessment | | | | | |

¹All patients who are capable of becoming pregnant must take a pregnancy test which is negative within 24 hours before beginning treatment.
²Inflammatory markers test will be performed at baseline and days 3, 8 and 11 during hospitalization and until discharge
³NovoTTF-100L device usage assessment will be performed only in the treatment arm of the study on day 15 of treatment and at the end of treatment
⁴RT-PCR SARS-CoV-2 OP swab test will be performed following discharge on days 15 and 29
⁵If clinically relevant

4. Analysis of Endpoints

The primary outcome can use an ordinal severity scale with 8 categories (defined below). Time to recovery, where recovery is defined as clinical status in states 1, 2, or 3 of the 8-point ordinal scale, censored at Day 29.

8-point ordinal scale: Not hospitalized, no limitations on activities; Not hospitalized, limitation on activities and/or requiring home oxygen; Hospitalized, not requiring supplemental oxygen—no longer requiring ongoing medical care; Hospitalized, not requiring supplemental oxygen—requiring ongoing medical care (COVID-19 related or otherwise); Hospitalized, requiring supplemental oxygen; Hospitalized, on non-invasive ventilation or high flow oxygen devices; Hospitalized, on invasive mechanical ventilation or ECMO; Death.

The primary endpoint can be achieved if time-to-recovery can be significantly lower in the TTFields plus the best SOC arm than in the best SOC alone arm. The statistical hypothesis can be tested by comparing Kaplan-Meier time-to-recovery curves of the two groups using a one-sided stratified log-rank test.

Day of recovery can be measured from the date of randomization to the date of the subject satisfies one of the following three categories from the ordinal scale: 1) Not hospitalized, no limitations on activities; 2) Not hospitalized, limitation on activities and/or requiring home oxygen; 3) Hospitalized, not requiring supplemental oxygen and no longer requires ongoing medical care.

Any subjects that are lost to follow-up or terminated early prior to an observed recovery can be censored at the day of their last observed assessment. Subjects who complete follow-up but do not experience recovery can be censored at the day of their Day 29 visit. All deaths within Day 29 (and prior to recovery) can be considered censored at 28 days.

Median estimates and confidence intervals from Kaplan-Meier curves by treatment arm can be presented. A table can present median time to event along with 95% confidence intervals overall for each treatment arm along with the hazard ratio estimate and log rank p-values.

Clinical status at specific time points can include evaluation of the clinical status score at Days 8, 15, 22, and 29.

The number and proportion of subjects along with 95% confidence intervals by category of clinical status can be presented by treatment arm at Days 8, 15, 22, and 29.

Duration of hospitalization can be defined as 1st day of hospitalization. Duration of Hospitalization can be measured from the 1st day of hospitalization to the day of discharge.

The Duration can be summarized in a table using medians and quartiles by treatment arm.

Incidence of all cause mortality can include the number and percentage of subjects who died by Day 15 and Day 29 presented by treatment arm (denominator for the percentages can be the number of subjects in the Safety population in each treatment arm). The 14- and 28-day mortality rate, which can consider the amount of follow-up time for each subject can be calculated and presented. Mortality through Day 29 can be measured as the time interval, in days, between randomization and death.

At the time of analysis, subjects who are lost to follow-up or withdrew consent or still ongoing in study can be censored at the last date when they were known to be alive before Day 29. Subjects who complete follow-up can be censored at the day of their Day 29 visit. Differences in time-to-event endpoints can be summarized with median estimates and confidence intervals from Kaplan-Meier curves by treatment arm can be presented. A table can present median time to event along with 95% confidence intervals overall for each treatment arm along with the hazard ratio estimate and log rank p-values.

The 15- and 29-day mortality rate is the proportion of subjects who are dead at 14 and 28 day, respectively. These can be derived from the Kaplan Meier estimates of the survival rates at the defined time point.

Incidence of ICU admission can be analyzed by treatment arm. The number of subjects that were admitted and incidence rate (and CI) can be reported.

Duration of ICU stay can be measured by the ICU admission date to the discharge date from ICU. This can only include subjects that were not admitted at enrollment. The duration can be summarized in a table using medians and quartiles by treatment arm.

Incidence of non-invasive ventilation or high-flow oxygen use can be defined as where the clinical status score is equal to 6.

The incidence of new non-invasive ventilation or high-flow oxygen use can be analyzed by treatment arm. The number of subjects using non-invasive ventilation or high-flow oxygen use and the incidence rate (including CI) can be reported.

Duration of non-invasive ventilation or high-flow oxygen use can be measured from the date of clinical status (based on the 8-point ordinal scale) score is equal to 6 to the date where the clinical status score is less than 6. The total duration can be the sum of all durations, regardless of whether the event occur consecutively or in disjoint intervals. The duration can be summarized in a table using medians and quartiles by treatment arm.

Incidence of invasive ventilation can be analyzed by treatment arm. The number of subjects using invasive ventilator and the incidence rate (and CI) can be reported.

Duration of invasive ventilation can be measured from the date of invasive ventilation initiation to the date invasive ventilation stops. The total duration can be the sum of all durations, regardless of whether the event occur consecutively or in disjoint intervals.

The duration can be summarized in a table using medians and quartiles by treatment arm.

The incidence of ECMO use can be analyzed by treatment arm. The number of subjects using ECMO and the incidence rate (and CI) can be reported.

Duration of ECMO use can be measured from the date of ECMO use initiation to the date ECMO use stops. The total duration can be the sum of all durations, regardless of whether the event occur consecutively or in disjoint intervals.

The duration can be summarized in a table using medians and quartiles by treatment arm.

The incidence of readmission to a hospital due to COVID-19 symptoms can be analyzed by treatment arm. The number of subjects that were readmitted and incidence rate (and CI) will be reported.

Saturation levels (including stability): Descriptive statistics including mean, median, standard deviation, maximum, and minimum values and change from baseline by time point can be summarized by treatment arm. Changes from baseline values can be presented in line graphs over time with mean and SD plotted by treatment arm.

The duration of body temperature >38° C. can be measured by the first date the patient has body temperature >38° C. after randomization to the date the body temperature decreases (<38° C.). The duration can be summarized in a table using medians and quartiles by treatment arm.

Level of body temperature: Descriptive statistics including mean, median, standard deviation, maximum, and minimum values and change from baseline by time point can be summarized by treatment arm. Changes from baseline values can be presented in line graphs over time with mean and SD plotted by treatment arm.

Supplemental oxygenation can be defined as where the clinical status (based on the 8-point ordinal scale) score is equal to 5, 6 or 7. Duration of supplemental oxygenation can be measured from the date of clinical status score is equal to 5, 6 or 7 to the date where the clinical status score is less than 5. The total duration can be the sum of all durations, regardless of whether the event occur consecutively or in disjoint intervals. The duration can be summarized in a table using medians and quartiles by treatment arm.

Inflammatory status is measured as the difference in CRP, D-dimer and ferritin blood levels from baseline on days 3, 8 and 15 until discharge between the two study arms. Descriptive statistics including mean, median, standard deviation, maximum, and minimum values and change from baseline by time point for each inflammatory biomarker can be summarized by treatment arm. Changes from baseline values can be presented in line graphs over time with mean and SD plotted by treatment arm.

The number and percentages of patients that have improvement in radiological assessment can be summarized by treatment arm.

### E. Example 5

1. Introduction

Coronaviruses are enveloped RNA viruses, with a single-stranded, positive-sense RNA genome. Their genome is complexed with the nucleocapsid (N) protein and encapsulated by a lipid bilayer with three structural proteins embedded therein: envelope (E), membrane (M), and spike (S). The S protein is constituted of 2 subunits, S1 that mediates viral binding to the host cell receptor, and S2 that promotes fusion of the viral envelope with the host cell membrane. As the crucial step in viral infection is entry of the virus into the host cells, therapeutic approaches to prevent infection are mostly focused on blocking the binding of the S protein to its receptor.

Binding affinity of viruses to the host receptors is mainly affected by electrostatic protein-protein interactions. In the case of Severe Acute Respiratory Syndrome Coronavirus 2 (SARS-CoV-2), the infectious agent responsible for the COVID-19 pandemic, host cell entry is mediated via interaction with the human angiotensin-converting enzyme 2 (ACE2) receptor. The positively charged S trimer in its closed conformation engages in transient nonspecific binding with the negatively charged ACE2 receptor, an interaction dominated by electrostatic forces. Next, the trimer rearranges to the open state, exposing its specific receptor-binding interface, and forming a complex that is stabilized by additional interactions. Binding affinity of the SARS-CoV-2 S protein to the ACE2 receptor was found to be higher than that of the earlier SARS-CoV strain, a phenomenon that has been attributed to the higher positive charge of the former, further underlining the importance of electrostatic interactions in coronavirus infection.

This important role of electrostatic interactions for viral infection suggests utilizing electric fields for interfering with viral host entry. A clinically approved treatment based on application of electric fields is Tumor Treating Fields (TTFields) [10]. TTFields therapy is applied to the thorax of oncological patients in the US and Europe for treatment of two pulmonary cancer indication, malignant pleural mesothelioma (approved indication, based on STELLAR trial) and non-small cell lung carcinoma (ongoing phase 3 study, LUNAR trial, NCT02973789). This potent cancer treatment modality is based on non-invasive delivery of low intensity (1-3 V/cm root-mean-square (RMS)), intermediate frequency (100-500 kHz), alternating electric fields via arrays placed on to the patient's skin at the tumor site. While electric fields at these frequencies are too high to stimulate nerve cells and too low to cause significant tissue heating, they are exactly adequate to penetrate cancerous cells and exert bi-directional forces therein. TTFields have been shown to cause alignment of the polar tubulin and septin molecules according to the electric field thus impairing their proper function during cell division, causing an anti-mitotic effect and subsequently cell death. The high charge on the S protein of coronaviruses, and specifically that of SARS-CoV-2, suggest a possible effect of TTFields on its functioning.

The goal of the current study was to examine the in vitro effect of TTFields on coronavirus infection and evaluate their safety in COVID-19 patients. Since the S protein, which is the major viral protein involved in viral attachment and entry into host cells, is highly conserved in all human coronaviruses, the less infectious member of this family, HCov-229E, was utilized in this study. Application of TTFields to human pulmonary cells was shown to lower viral entry and replication. Furthermore, progeny virions formed under TTFields displayed lower infectivity. TTFields were also shown to enhance the in vitro efficacy of remdesivir, an authorized treatment for COVID-19 patients with severe disease. Lastly, the safety of applying TTFields to COVID-19 patients concomitant with remdesivir was demonstrated.

2. Methods

i. Cell line and Virus.

Human MRC-5 lung fibroblast cells (ATCC, CCL-171™) and human lung carcinoma cell line A549 (ATCC, CCL-185™) were grown in 5% CO2 humidified incubator at 37° C. in Eagle's Minimum Essential Medium (EMEM) (ATCC, 30-2003™) and Dulbecco's Modified Eagle's medium (DMEM) (Biological Industries, 01-055-1A), respectively, supplemented with 10% fetal bovine serum (FBS) (Biological Industries, 04-007-1A). HCoV-229E (ATCC, VR740™) was handled in Biosafety level 2 (BSL2) facilities and grown at its optimal temperature of 35° C. throughout. For production of a stock virus pool, the commercial HCoV-229E was grown in MRC-5 cells according to the supplier instructions, quantified by plaque assay, and stored in aliquots at −80° C.

ii. TTFields Application.

TTFields were applied using the Inovitro™ system (Novocure, ISR). Cell suspensions were grown in Inovitro™ dishes composed of high dielectric constant ceramic (lead magnesium niobate-lead titanate [PMN-PT]), with two perpendicularly pairs of transducers printed on their outer walls. The transducers were connected to a sinusoidal waveform generator that produces alternating electric fields at selected frequency and intensity, while changing field orientation perpendicularly every 1 s. For generating TTFields at an intensity of 1.5 V/cm RMS incubator temperature was set to 18° C. so that the resultant temperature within the dishes was 35° C.

iii. Effect of TTFields on Viral Entry.

MRC-5 cells were seeded on glass cover slips (22 mm diameter) at a density of 1.5×10⁵ cells/cover slip. After 24 h, the cells were transferred into inovitro dishes containing 2 ml EMEM supplemented with 2% FBS. The cells were then exposed to TTFields at a frequency range of 100 to 400 kHz, and 30 min later infected with HCoV-229E at multiplicity of infection (MOI) of 0.01 with continued TTFields application. Control cells were not treated with TTFields at any time. At 0.5 or 2 h post infection (hpi) the cells were washed with PBS, trypsinized, resuspended and counted using Scepter™ 2.0 Cell Counter (Merck, Millipore). Then the cells were washed with PBS and frozen at −80° C. until RT-qPCR analysis.

iv. Effect of TTFields on Long Term Viral Exposure and on Viral Replication.

MRC-5 cells were seeded, grown, and exposed to TTFields (150 kHz) as described above, and then infected with HCoV-229E at MOI of 0.0001. At 3 hpi the cells were washed with PBS to remove unbound viruses and maintained in fresh media for a total of 24, 48 or 72 h. TTFields were applied throughout starting from 30 min before infection, while control cells were not exposed to TTFields at any time. At treatment end, growth media were collected and stored at −80° C. for RT-qPCR analysis and plaque assay. The cells were processed as described above. The same procedure was undertaken with A549 cells, seeding 1.0×10⁵ cells/cover slip. Alternatively, MRC-5 cells were infected with HCoV-229E at MOI of 0.01, the cells were washed at 3 hpi, and only then TTFields were applied for up to 24 hpi, followed by analysis of dsRNA formation.

v. The Combined Effect of TTFields with Remdesivir.

MRC-5 cells were seeded, grown, and exposed to TTFields (150 kHz) as described above, and then infected with HCoV-229E at MOI of 0.01. At 3 hpi the cells were washed with PBS and maintained with or without application of TTFields in fresh media to which 0, 0.011, or 0.023 μM remdesivir (Cayman Chemicals, Cay30354) were added. At 48 hpi, growth media and cells were collected and analyzed as described above.

vi. Real-Time Quantitative Reverse Transcription PCR (RT-qPCR).

Total RNA was extracted using the MagnaPure 96 instrument (Roche, Germany) according to the manufacturer instructions. Reactions were performed in 25 μl reaction mixtures, prepared with AgPath-ID™ One-Step RT-PCR Reagents (Applied Biosystems, Thermo Fisher Scientific) using type-specific primers and probes (Hylabs, Israel) for HCoV-229E selected with Primer Express software (PE Applied Biosystems) based on the genomic regions of high conservation of the nucleocapsid gene: Forward: 5′-CAGTCAAATGGGCTGATGCA-3′; Reverse: 5′-AAAGGGCTATAAAGAGAATAAGGTATTCT-3′; Probe: 5′-CCCTGACGACCACGTTGTGGTTCA-3′, 5′ labelled with fluorescein amidite (FAM). Amplification and detection were performed using TaqMan Chemistry on the ABI 7500 instrument with the following conditions: 48° C. for 30 min (1 cycle); 95° C. for 10 min (1 cycle); and 95° C. for 10 s followed by 60° C. for 1 min (45 cycles). The amount of HCoV-229E in the supernatant (SN) was quantified per volume and expressed as percent relative to control. To determine intracellular HCoV-229E, relative quantification (RQ) was employed using RNaseP as the cellular normalizing gene: Forward: 5′-AGATTTGGACCTGCGAGCG-3′; Reverse: 5′-GAGCGGCTGTCTCCACAAGT-3′; Probe: 5′-TTCTGACCTGAAGGCTCTGCGCG-3′, 5′ labelled with FAM.

vii. Scanning Electron Microscopy (SEM).

MRC-5 cells were seeded on glass coverslips (13 mm diameter) at a density of 4×10⁴ cells/cover slip, and handled as described above. The cells were infected with the HCoV-229E virus at MOI of 20. TTFields (150 kHz) were applied throughout, starting from 30 min before infection, while control cells were not treated with TTFields. At 0.5 hpi the slides were transferred to clean plates, washed briefly with PBS and fixed using 2% glutaraldehyde and 1% paraformaldehyde in 0.1 M sodium cacodylate buffer for 2 h at room temperature. Following 15 min fixation with osmium tetroxide in cacodylate buffer, the samples were dehydrated in graded ethanol series, critical point dried (Quorum K850) and sputter coated with 4 nm of iridium (Quorum Q150T). Samples were then viewed on Zeiss Ultra Plus HR Scanning Electron Microscope.

viii. Transmission Electron Microscopy (TEM).

MRC-5 cells were seeded on thermanox coverslips (22 mm diameter) (Thermo, 174977) at a density of 3×10⁴ cells/coverslip. The cells were then grown as described above, infected with HCoV-229E at MOI of 0.03, washed at 3 hpi, and only then TTFields were applied for up to 48 hpi. Control cells were not treated with TTFields at any time. Then the cells were fixed for 2 h with 2% glutaraldehyde, 3% paraformaldehyde, in 0.1 M sodium cacodylate buffer containing 5 mM CaCl2. The samples were washed, post fixed using 2% osmium tetroxide, washed with DDW and incubated in 2% uranyl acetate. Following dehydration in graded ethanol series, the coverslips with the cells were moved to fresh wells filled with Epon812 for embedding. 75 nm transverse sections were cut using ultramicrotome UC7 (Leica), transferred to copper grids and viewed using Talos L120C Transmission Electron Microscope at accelerating voltage of 120 keV.

ix. Immunofluorescence Imaging of dsRNA.

Cells were fixed with ice-cold absolute ethanol (Millipore, 100983) for 15 min followed by three washes with PBS. The cells were then blocked in 1% BSA (Sigma, A7906) diluted in PBS for 30 min, incubated with anti-double stranded RNA monoclonal antibody (SCICONS J2, 10010200) diluted 1:100 in PBS containing 1% BSA for at least 1 hr at room temperature, followed by 3 washes with PBS. Next, the cells were incubated with IgG Alexa fluor 488-conjugated donkey anti mouse antibody diluted 1:800 in PBS containing 1% BSA and 1 μg/ml 4′,6-diamidino-2-phenylindole (DAPI) (Sigma, 32670) for 40 min, washed 3 times with PBS, and mounted to slides. Images were collected using LSM 700 laser scanning confocal system (Zeiss Gottingen). Image analysis and quantification were done using the FIJI software.

x. Plaque Assay.

MRC-5 cells were seeded in 12 well plates at a density of 2×105 cells/well. After 24 hr, the incubator temperature was changed to 35° C., the medium was replaced with fresh 1 ml EMEM supplemented with 2% FBS, and the cells were infected with the supernatant from the 48 hr long-term viral exposure experiments, 1.9×105 virus copies per well and 5 serial 10-fold dilutions. At 2 hpi, the cells were washed with PBS to remove unbound viruses and covered with EMEM supplemented with 2% FBS and 1.5% carboxymethylcellulose (CMC) (Sigma C4888). Four days later the cells were fixed with ice-cold absolute ethanol for 15 min and stained with 1% Cristal Violet (Mercury, 1159400025), 20% ethanol for 5 min at room temperature. The plaque forming unites (PFU) were counted and divided by the dilution factor for obtaining the PFU per equal virus amount. To calculate the PFU for equal supernatant (SN) volumes the following equation was applied: number of plaques formed by identical virus dilutions/dilution factor×infection volume of SN in 1 ml infection media.

xi. Statistical Analysis

Each in vitro experiment was repeated for three times and the data from all repeats were pooled for the analysis and are presented as means±SD. Statistical significance was calculated using GraphPad Prism 8 software (La Jolla), with the specific tests used mentioned in figure legends. Differences were considered significant at values of: *p<0.05, **p<0.01, ***p<0.001, and ****p<0.0001.

xii. Safety Clinical Trial.

EF-37 study was a single arm, open label pilot trial of NovoTTF-100L (150 kHz TTFields) on hospitalized patient with COVID-19 disease.

Overall 10 patients were recruited. Eligible for the study were hospitalized patients that were found positive to COVID-19. Inclusion criteria were: age >18; hospitalized with diagnosis of COVID-19 infection per RT-PCR within 72 hr prior to treatment start; SpO2<93% at sea level; lung involvement confirmed with chest imaging; able and willing to comply with all study procedures; and for female participants of childbearing age use of highly effective contraception (a failure rate <1% per year when used consistently and correctly). Exclusion criteria included: receipt of any experimental treatment for COVID-19 prior to or during the study; assisted ventilation; critical illness, defined as respiratory failure (SpO2/FiO2<150), septic shock, and/or multiple organ dysfunction; significant co-morbidities at baseline including clinically significant hematological, hepatic and renal dysfunction (defined as neutrophil count <1.5×109/L and platelet count <100×109/L, bilirubin >1.5×ULN, AST and/or ALT>2.5×ULN, and serum creatinine >2.5 mg/dL), history of significant cardiovascular disease unless the disease is well controlled (second/third degree heart block, significant ischemic heart disease, poorly controlled hypertension, congestive heart failure, or symptoms of heart failure at rest), history of arrhythmia that is symptomatic or requires treatment, or history of any psychiatric condition that might impair patient's ability to understand or comply with the requirements of the study or to provide consent; implantable electronic medical devices (e.g. pacemaker, defibrillator) in the upper torso; pregnancy or breast-feeding; known allergies to medical adhesives or hydrogel; or unwilling or unable to comply with the requirements of this protocol.

Written informed consent was obtained from all patients prior to any study related assessments/procedures being conducted. Enrolment was performed within 48 hr of hospitalization, and patients were treated with TTFields delivered to the thorax (>18 hr/day) while receiving concomitant treatment with COVID-19 standard of care for a duration of 29 days or until not hospitalized any more. Clinical follow-up continued for 30 days post treatment end. The safety profile was measured by the frequency and severity of treatment associated adverse events based on the Common Terminology Criteria for Adverse Events (CTCAE) V5.0. Secondary endpoints included: all-cause mortality, incidence of Intensive Care Unit (ICU) admission, incidence of non-invasive ventilation or high-flow oxygen use, incidence of invasive ventilation, incidence of extracorporeal membrane oxygenation (ECMO), patient inflammatory status, duration of hospitalization, time to recovery, and clinical status at day 8, 15, 22, 29 of treatment.

TTFields were delivered to patients through four insulated surface arrays, placed on the patients' skin surrounding the thorax as to generate two perpendicular fields in the chest of the patient. The area where the arrays were planned to be applied was shaved if needed, a layer of adhesive hydrogel was positioned beneath the arrays, and hypoallergenic medical tape placed on top of the arrays. The arrays were replaced two to three times a week in order to maintain optimal coupling between the transducer arrays and the patients' skin. The arrays were attached to a field generator delivering currents of 1414 mA at two sequential, perpendicular directions. The NovoTTF-100A's internal memory unit captured the time TTFields were delivered, thereby allowing objective usage reports. Treatment was given continuously for at least 18 hr per day throughout the study.

3. Results

i. Effect of TTFields on Viral Entry

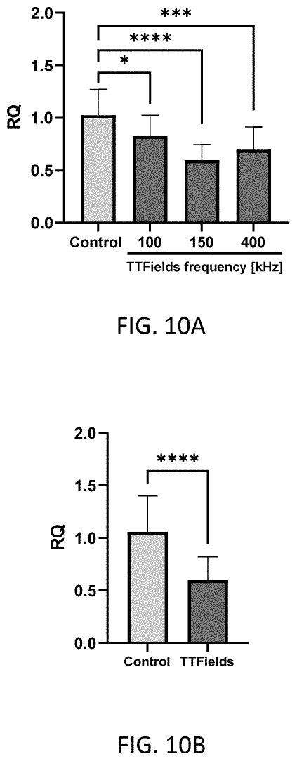

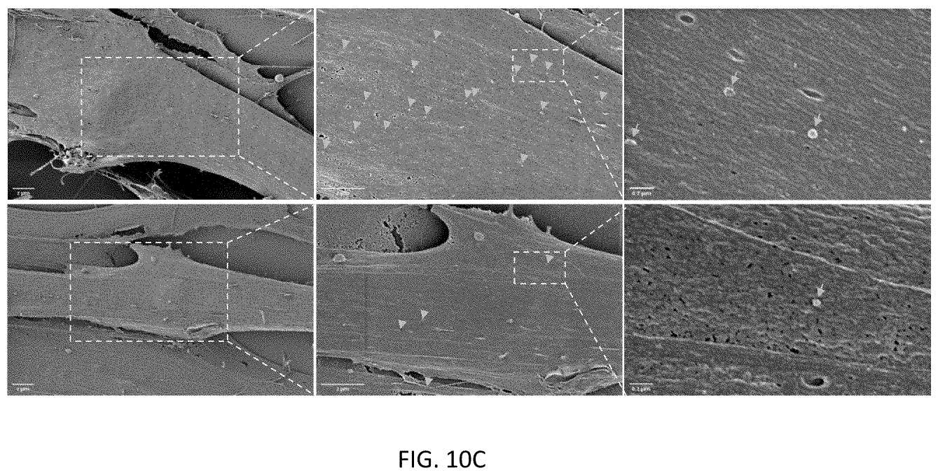

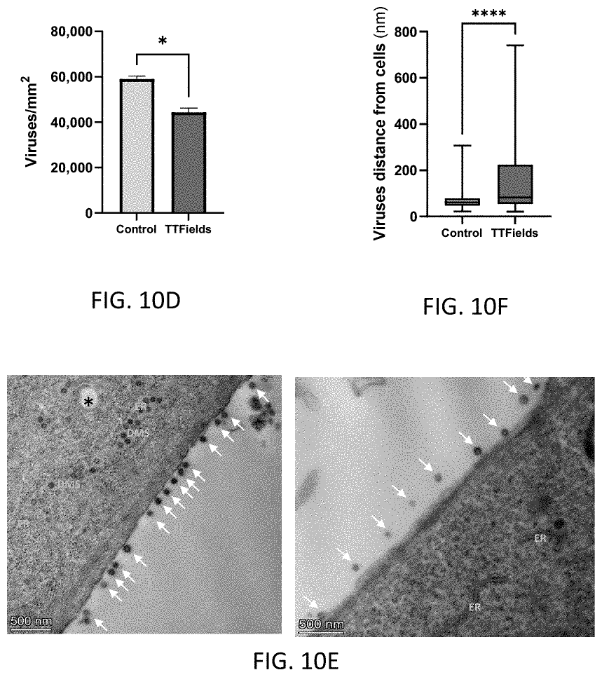

Application of 150 kHz TTFields to MRC-5 lung fibroblasts during 2 h of infection with HCoV-229E at MOI of 0.01 produced a significant 42% reduction in cellular viral load relative to infected cells with no treatment (FIG. 10A). Frequencies of 100 and 400 kHz TTFields were less effective, displaying reductions of 19 and 32%, respectively. Accordingly, the TTFields frequency of 150 kHz was selected for all consecutive experiments. Similar experiments performed with only 30 min viral incubation of only 30 min as to focus on the cellular attachment step showed 43% inhibition for TTFields relative to control (FIG. 10B).

To further examine the effect of TTFields on viral cellular attachment, SEM and TEM examinations were performed. SEM was utilized on MRC-5 cells exposed for 30 min to HCoV-229E at a high MOI of 20 as to allow easier visualization of the virions, showing a 25% reduction in the number of virions bound to the cell surface when TTFields were applied relative to control (FIG. 10C and FIG. 10D). TEM examinations performed at 48 hpi on control cells infected for 3 h with HCoV-229E at MOI of 0.03 revealed that the average distance of the virions from the cells was 75 nm (range=21-307 nm), whereas when TTFields were applied during the time period following infection the average distance elevated to 158 nm (range=21-740 nm) (FIG. 10E and FIG. 10F).

ii. Effect of TTFields on Long-Term Viral Exposure

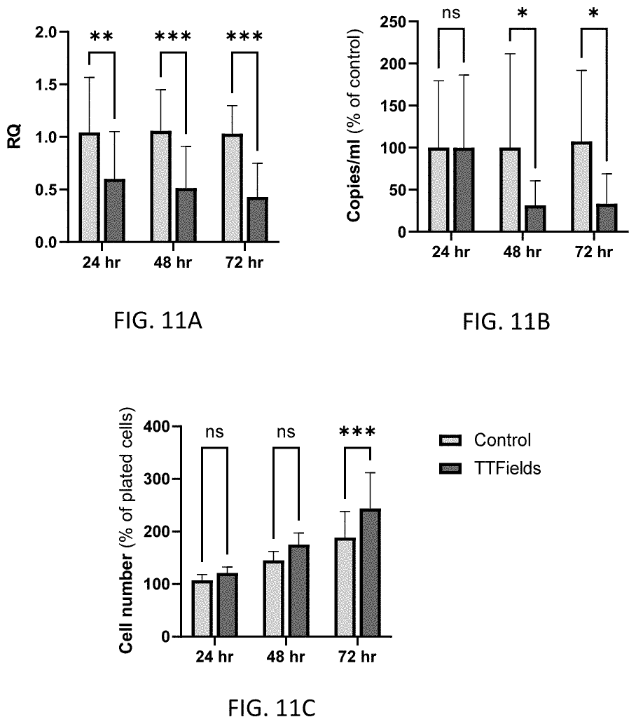

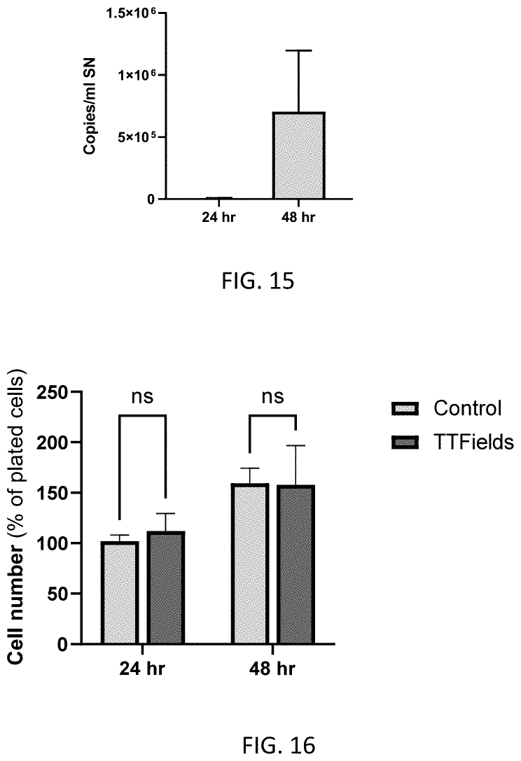

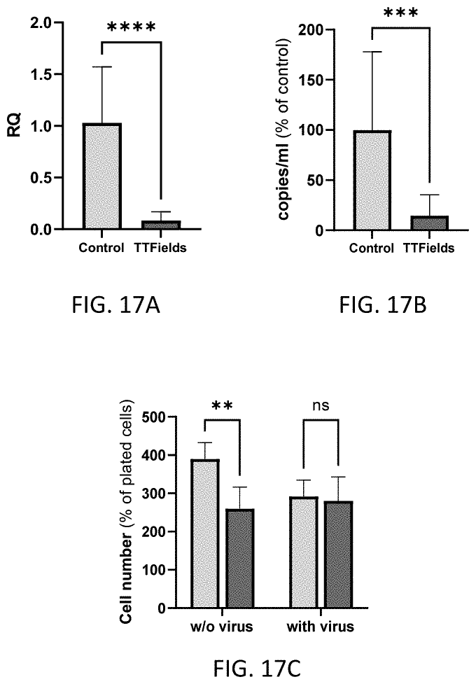

MRC-5 cells infected with HCoV-229E at MOI of 0.0001 while being exposed to 150 kHz TTFields during infection and up to 24, 48 or 72 hpi, showed reduced viral cellular load relative to control by 42, 51, and 58%, respectively (FIG. 11A). The number of viruses secreted to the media was also affected by TTFields, with 68 and 74% reduction relative to control for 48 and 72 hr delivery of TTFields, respectively (FIG. 11B). Within 24 hours of infection very low levels of virus were secreted from the cells (FIG. 15), and hence no significant difference was seen between control and TTFields regarding the extracellular viral amount at this timeframe. While infected MRC-5 cells displayed lower cell counts than non-infected cells in the absence of TTFields, 9% at 48 hpi, and by X % at 72 hpi (FIG), wherein TTFields were applied to infected cells the number of cells elevated by 19, 21, and 35% relative to control cells for 24, 48, and 72 hr treatment, respectively (FIG. 11C). Cell count was, however, not affected by delivery of TTFields to the cells in the absence of the virus (FIG. 17C), altogether indicating that TTFields were protecting the cells from the deleterious effects of the virus. The supernatants from the 48-h long-term viral exposures were subjected to plaque forming assays in MRC-5 cells, without further application of TTFields. Viral titer formed under application of TTFields was 79% lower than that formed without TTFields when examining equal supernatant volumes (FIG. 11D), while PFU per equal amounts of viruses was lower by 50% (FIG. 11E), indicating a difference not only in viral quantity but also in its virulence.

The effect of TTFields on infection of A549 lung cancer cells was also examined. These cells showed higher sensitivity to the virus relative to MRC-5 cells, with 25% decrease in cell counts for HCoV-229E-infected (MOI of 0.0001) versus non-infected A549 cells at 48 hpi (FIG. 17B). Nevertheless, the protective effect of 150 kHz TTFields also elevated in these cells, with intracellular viral load reduced by 92% (FIG. 17A), and the number of extracellular viral copies decreased by 85% (FIG. 17C). It should be noted that 150 kHz TTFields are known to be cytotoxic to A549 cells as part of the antimitotic effect of TTFields on cancerous cells. Indeed, A549 cells exposed to 150 kHz TTFields for 48 hr, without viral infection, displayed 33% lower cell count relative to control (FIG. 17C). However, no difference in cell count was seen for cells exposed to TTFields for 48 hr in the presence of the virus relative to control.

iii. Effect of TTFields on Viral Replication

MRC-5 cells were infected with HCoV-229E at MOI of 0.01 for 3 hr, the cells were then washed to remove any extracellular virions, and only then TTFields were applied for up to 24 hpi, a time frame in which secretion of virions to the media is scarce (FIG. 15), and thus minimal levels of re-infection events are expected. This protocol allowed isolating the effect of TTFields on the replication step which was measured by fluorescent microscopy for detection of dsRNA, associated with viral replication (FIG. 12A). As the infection step was performed identically, without delivery of TTFields, the number of infected cells was equal in both groups (not shown). However, there was a 24% reduction in the number of foci per infected cell for application of TTFields versus control (FIG. 12B), as well as a 23% decrease in foci size (FIG. 12C), and a decline of 41% in the overall foci area per infected cell (FIG. 12D). TEM examinations were performed as to allow examination of intracellular events (FIG. 12E and FIG. 12H). Cells that were infected for 3 h with HCoV-229E at MOI of 0.03 and examined at 48 hpi while being exposed to TTFields displayed 70% less double membrane vesicles (DMVs) invaginations (FIG. 12F), and 98% less cases of DMV fusions (FIG. 12G). On the other hand, 3-fold higher levels of autophagolysosomes were seen in infected cells that were treated with TTFields relative to the control cells (FIG. 12I, FIG. 12H).

The supernatants from the 48 hr long-term viral exposures were subjected to plaque forming assays in MRC-5 cells, without further application of TTFields. When examining equal amounts of viruses formed with or without application of TTFields, the former showed 44% lower plaque forming ability (FIG. 12I); and a 71% reduction when calculating the PFU per equal supernatant volumes (FIG. 12K).

iv. The Combined Effect of TTFields with Remdesivir

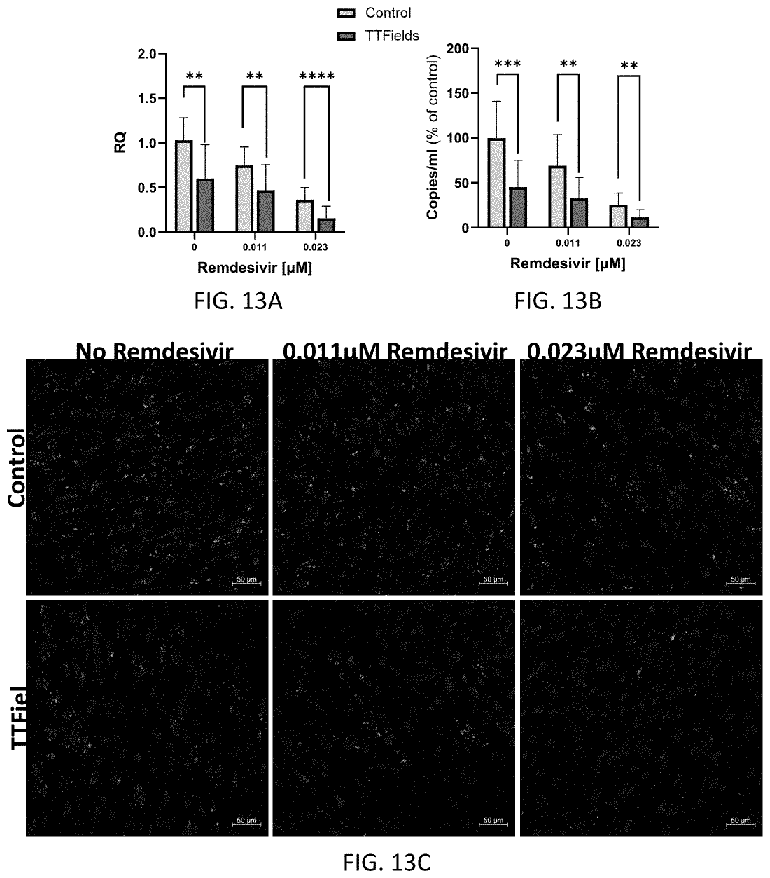

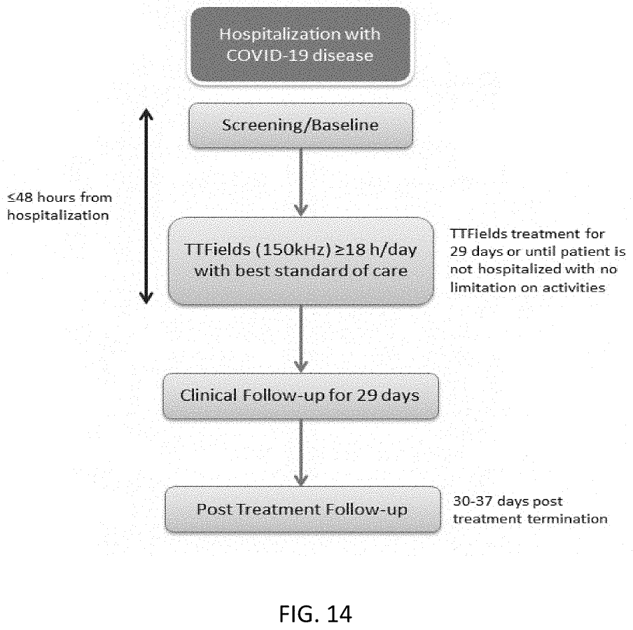

MRC-5 cells infected for 3 hr with HCoV-229E at MOI of 0.01 followed by treatment with remdesivir displayed dose dependent response, with 27% inhibition of cellular viral load for 0.011 μM remdesivir and 65% reduction for 0.023 μM remdesivir at 48 hpi (FIG. 13A). Delivery of TTFields alone under these conditions reduced viral load by 42%, while concomitant application of TTFields with remdesivir reduced viral load by 54% for the low dose and by 85% for the high dose. The lower viral load for co-application of TTFields with remdesivir was also evident from reduced levels of dsRNA within the cells relative to the mono-therapies (FIG. 13C). The number of virions secreted to the media was also reduced by 31% and 75% for 0.011 and 0.023 μM remdesivir, respectively, and by 55% for TTFields alone (FIG. 13B). Concomitant application of the two treatments resulted in a decrease of 68% for the low dose and 88% for the high dose.

The calculated additive effect was calculated by multiplying Remdesivir alone with TTFields alone. When comparing calculated to measured effect, in the higher concentration (0.023) there is a small additive effect (see Table 2).

TABLE 2

| Remedesivir | Remdesivir | TTFields | Measured | Calculated |
|---|---|---|---|---|
| concentration | alone | alone | combined | additive |
| 0.011 | 73% | 58% | 46% | 42% |
| 0.023 | 35% | 58% | 15% | 20% |

4. Discussion

In the current study the effect of TTFields on pulmonary infection by coronavirus was examined, using the less infectious strain HCoV-229E. TTFields were examined at several frequencies, with all showing significant inhibition of viral entry to MRC-5 lung fibroblasts, as determined from RT-qPCR measurements. The effect of 150 kHz was found to be most profound, and thus was employed throughout. The same level of inhibition by TTFields was detected for both 2 h and 30 min of viral infection, suggesting that TTFields mainly interfere with viral attachment rather than with the cellular internalization step. SEM and TEM examinations allowed visualization of the virions, with the former showing less attachment of virions to the cells, and the latter demonstrating a higher average distance of the virions from the cells, when TTFields were applied. Overall, these results indicate that TTFields interfere with viral attachment to the cells, leading to reduced viral entry.

As the virus life cycle includes secretion of progeny virions from the cells and repeated entry, the long-term effect of TTFields was also examined. In these 72 h long examinations TTFields reduced viral intracellular load effectively already after 24 h, with an increased effect for longer treatment durations. This was accompanied by a decrease in secretion of virions from the cells to the media, as evident from both RT-qPCR measurements and plaque assays. Furthermore, cell counts were higher than control for cells treated with TTFields, with the effect elevating for a longer treatment time, showing that TTFields were protecting the cells from the harmful consequences of viral infection. Long-term examinations were also performed in A549 lung carcinoma cells, in which a profound decrease in cellular viral load and in viral secretion to the media were observed for cells treated with TTFields.

Application of electric fields have been shown to affect the secondary and tertiary structure of the SARS-CoV-2 S protein. Simulations revealed alignment of local dipoles and displacement of charges parallel to the fields orientation, resulting in disruption of the spatial organization of key residues involved in receptor binding in a non-reversable manner, when electric fields at a low intensity of 100 V/cm were delivered for a few hundred nanoseconds. This structural alternation of the S protein following exposure to electric fields was suggested to cause inactivation or attenuation of the virions. While TTFields utilized in the current study were only at an intensity of 1.5 V/cm RMS, intensity amplification has been shown to occur in the vicinity of the cellular membrane due to its non-permittivity.

In depth examination of the plaque assays from the long-term experiments revealed an interesting outcome—PFU for virions formed under application of TTFields was lower than control not only for equal supernatant volumes, but also for identical amounts of virus. This indicates that progeny virions formed under TTFields were less virulent than control. One explanation for this phenomenon can be conformational changes of S protein induced by the electric fields, as described above. A second option is associated with tubulin, that has been suggested to be involved in S protein transport, localization, and assembly into virions, with reduced release of infectious virus particles observed when microtubule depolymerization was induced. TTFields have been shown to mediate changes in microtubules organization and dynamics in cancerous cells. This effect of TTFields can be relevant in non-cancerous cells and hence interferes with proper assembly of virions. TTFields can interfere with additional steps of the viral life cycle.

To examine effects of TTFields on viral replication, cells were first infected with HCoV-229E, and only then TTFields were delivered. As the infection step was done identically in both control and treatment groups, no differences were seen in the number of infected cells. However, the amount and size of dsRNA foci formed within infected cells when TTFields were applied was lower than control; and there were fewer DMV invagination and fusion events. On the other hand, higher amounts of autophagolysosomes (the result of fusion of autophagosomes with lysosomes) were seen in the cells exposed to TTFields, indicative of elevated autophagic flux in these cells. Cells infected with coronavirus are known to utilize the autophagy pathway to sense, control the growth, and clear the virus. Viruses have evolved to inhibit, escape, or manipulate this host response to protect themselves. In the case of coronavirus, upon infection the virus hijacks the autophagosomes, preventing their fusion with the lysosomes, and utilizing these DMVs as replication and translation niches. At later stages, DMV invagination and fusion allow repurposing of membranes for further virion production. Altogether the results demonstrate that TTFields interfere with viral replication, what may be attributed at least in part to the ability of TTFields to elevate autophagy, as previously demonstrated in glioblastoma, Lewis lung carcinoma, and hepatocellular carcinoma cell lines.

On May 1, 2020, the FDA issued an emergency use authorization for use of remdesivir as treatment for COVID-19 patients with severe disease, as the potential benefits of this drug were determined to outweigh its known and potential risks. The possible combination of TTFields with remdesivir was examined, shown in vitro to be superior to each treatment alone regarding both viral intracellular accumulation and secretion. This indicates that lower remdesivir doses can be sufficient for concomitant treatment with TTFields, and thus, may allow alleviation of the remdesivir associated risks. The safety of combining TTFields and remdesivir was examined in a phase 1 clinical trial.

To conclude, the efficacy of TTFields for preventing coronavirus infection and replication, as well as the safety of applying TTFields to COVID-19 patients, have been demonstrated. As the COVID-19 pandemic continues to spread rapidly around the globe, the virus continues to mutate in a fast pace. Changes in pivotal residues at the S protein result in variants that display enhanced viral-host cells attachment and fusion and hence increased infectivity and reduced treatability of these new forms of the virus. This may be seen with the more contagious variants of the current spreads, the Delta (B.1.617.2 lineage) and Lambda (C.37 lineage) variants, that host multiple mutations in the S protein receptor binding domain, and display relative resistance to the neutralizing antibodies that are elicited in convalescent and vaccinated individuals. These changes can also hinder the effectivity of therapeutic approaches, that are mainly dependent on the structure of the S protein and its interaction with the host receptor. As TTFields are not tailored against any specific S protein amino acids sequence, but rather to the high protein polarity responsible for increased host receptor binding, they can be suitable for treatment of different viral variants, suggesting promise for this treatment modality in the everchanging viral landscape.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Wang, D., et al., Clinical Characteristics of 138 Hospitalized Patients With 2019 Novel Coronavirus-Infected Pneumonia in Wuhan, China. JAMA, 2020.
2. Lu, H., C. W. Stratton, and Y. W. Tang, Outbreak of pneumonia of unknown etiology in Wuhan, China: The mystery and the miracle. J Med Virol, 2020. 92(4): p. 401-402.
3. van Doremalen, N., et al., Aerosol and Surface Stability of SARS-CoV-2 as Compared with SARS-CoV-1. N Engl J Med, 2020. 382(16): p. 1564-1567.
4. Lai, C. C., et al., Severe acute respiratory syndrome coronavirus 2 (SARS-CoV-2) and coronavirus disease-2019 (COVID-19): The epidemic and the challenges. Int J Antimicrob Agents, 2020. 55(3): p. 105924.
5. Kirson, E. D., et al., Alternating electric fields arrest cell proliferation in animal tumor models and human brain tumors. Proc Natl Acad Sci USA, 2007. 104(24): p. 10152-7.
6. Kirson, E. D., et al., Disruption of cancer cell replication by alternating electric fields. Cancer Res, 2004. 64(9): p. 3288-95.
7. Stupp, R., et al., Effect of Tumor-Treating Fields Plus Maintenance Temozolomide vs Maintenance Temozolomide Alone on Survival in Patients With Glioblastoma: A Randomized Clinical Trial. JAMA, 2017. 318(23): p. 2306-2316.
8. Stupp, R., et al., NovoTTF-100A versus physician's choice chemotherapy in recurrent glioblastoma: a randomised phase III trial of a novel treatment modality. Eur J Cancer, 2012. 48(14): p. 2192-202.
9. Ceresoli, G. L., et al., Tumour Treating Fields in combination with pemetrexed and cisplatin or carboplatin as first-line treatment for unresectable malignant pleural mesothelioma (STELLAR): a multicentre, single-arm phase 2 trial. Lancet Oncol, 2019. 20(12): p. 1702-1709.
10. Gera, N., et al., Tumor treating fields perturb the localization of septins and cause aberrant mitotic exit. PLoS One, 2015. 10(5): p. e0125269.
11. Giladi, M., et al., Mitotic Spindle Disruption by Alternating Electric Fields Leads to Improper Chromosome Segregation and Mitotic Catastrophe in Cancer Cells. Scientific Reports, 2015. 5: p. 18046.
12. Karanam, N. K., et al., Tumor treating fields cause replication stress and interfere with DNA replication fork maintenance: Implications for cancer therapy. Transl Res, 2020. 217: p. 33-46.
13. Voloshin, T., et al., Tumor-treating fields (TTFields) induce immunogenic cell death resulting in enhanced antitumor efficacy when combined with anti-PD-1 therapy. Cancer Immunol Immunother, 2020.
14. Porat, Y., et al., Determining the Optimal Inhibitory Frequency for Cancerous Cells Using Tumor Treating Fields (TTFields). J Vis Exp, 2017(123).
15. Ballo, M. T., et al., Correlation of Tumor Treating Fields Dosimetry to Survival Outcomes in Newly Diagnosed Glioblastoma: A Large-Scale Numerical Simulation-Based Analysis of Data from the Phase 3 EF-14 Randomized Trial. Int J Radiat Oncol Biol Phys, 2019. 104(5): p. 1106-1113.

We claim:

1. A method of inhibiting a coronavirus from infecting or replicating in a cell comprising:
exposing the cell to alternating electric fields for a period of time, the alternating electric fields having a frequency and field strength,
wherein the frequency and field strength of the alternating electric fields inhibits coronavirus infection or replication.

1. The method of claim 1, wherein the cell is in a subject.

2. The method of claim 1, wherein the coronavirus is SARS-CoV-2.

3. The method of claim 1, wherein the alternating electric fields have a frequency of between 50 kHz and 1 MHz.

4. The method of claim 1, wherein the alternating electric fields has a field strength of at least 1 V/cm RMS.

5. The method of claim 1, wherein the alternating electric fields are multidirectional.

6. A method of reducing coronavirus copy number per cell comprising:
exposing the cell to alternating electric fields for a period of time, the alternating electric field having a frequency and field strength,
wherein the frequency and field strength of the alternating electric fields reduces virus copy number in the cell.

7. The method of claim 7, wherein the cell is in a subject.

8. The method of claim 7, wherein the coronavirus is SARS-CoV-2.

9. The method of claim 7, wherein the alternating electric fields have a frequency of between 50 kHz and 1 MHz.

10. The method of claim 7, wherein the alternating electric fields have a field strength of at least 1 V/cm RMS.

11. The method of claim 7, wherein the alternating electric fields are multidirectional.

12. A method of treating a subject infected with coronavirus comprising:
applying alternating electric fields to a target site of the subject for a period of time, the alternating electric fields having a frequency and field strength,
wherein the target site comprises one or more coronavirus infected cells.

13. The method of claim 13, wherein the coronavirus is SARS-CoV-2.

14. The method of claim 13, wherein the alternating electric fields are applied at a frequency of between 50 kHz and 1 MHz.

15. The method of claim 13, wherein the target site of the subject is the lungs.

16. The method of claim 13, wherein cells not infected with a coronavirus are not damaged.

17. The method of claim 13, wherein the alternating electric fields have a field strength of at least 1 V/cm RMS.

18. The method of claim 13, wherein the viability of the cells at the target site is maintained and viral replication or viral infection is decreased.

19. The method of claim 13, wherein the alternating electric fields are multidirectional.
